(19) United States
(12) Patent Application Publication
(10) Pub. No.: US 2025/0152971 A1
(43) Pub. Date: May 15, 2025

(54) EXPLORATION OF PARETO-OPTIMAL RADIOTHERAPY PLANS

(71) Applicant: Elekta Instrument AB, Stockholm (SE)

(72) Inventors: Jens Olof Sjolund, Uppsala (SE); Carl Axel Håkan Nordström, Stockholm (SE)

(21) Appl. No.: 18/832,749

(22) PCT Filed: Feb. 11, 2022
(86) PCT No.: PCT/EP2022/053440

§ 371 Date: Jul. 24, 2024

(51) Int. Cl. A61N 5/10; G16H 20/40

(52) U.S. Cl. CPC A61N 5/1031; A61N 5/1084; G16H 20/40; A61N 2005/1035; A61N 2005/1074

(57) ABSTRACT

Systems and methods are disclosed for exploration and adaptation of radiotherapy treatment plans. Example operations for radiotherapy treatment planning include: obtaining a plurality of solutions (e.g., Pareto-optimal solutions) of a radiotherapy problem, exploring the plurality of solutions to identify an additional solution in a submanifold space (e.g., exploration of a Pareto surface), and generating treatment plan parameters based on the additional solution for use in a radiation therapy treatment. In an example, exploring the plurality of solutions includes: establishing a submanifold space from a manifold space representing the plurality of solutions in fewer dimensions than the weights; producing additional sets of weights in the submanifold space based on derivatives of first-order optimality conditions of the radiotherapy problem, the derivatives determined with respect to the weights; and navigating in the submanifold space to arrive at the additional solution, corresponding to one of the additional sets of weights.

### TECHNICAL FIELD

Embodiments of the present disclosure pertain generally to processing and optimization techniques used in connection with a radiation therapy planning and treatment system. In particular, the present disclosure pertains to methods and the use of specific computing hardware configurations to navigate a multitude of Pareto-optimized radiotherapy plans.

### BACKGROUND

Radiation therapy (or “radiotherapy”) can be used to treat cancers or other ailments in mammalian (e.g., human and animal) tissue. One such radiotherapy technique is provided using a Gamma Knife, by which a patient is irradiated by a large number of low-intensity gamma rays that converge with high intensity and high precision at a target (e.g., a tumor). Another such radiotherapy technique is provided using a linear accelerator (Linac), whereby a tumor is irradiated by high-energy particles (e.g., electrons, protons, ions, high-energy photons, and the like). The placement and dose of the radiation beam must be accurately controlled to ensure the tumor receives the prescribed radiation, and the placement of the beam should be such as to minimize damage to the surrounding healthy tissue, often called the organ(s) at risk (OARs).

In radiotherapy, treatment plans are usually generated by solving an optimization problem that balances various conflicting objectives, such as high dose to a target tumor, sparing healthy tissue, and other treatment complexities. Therefore, the optimization problem is a multicriteria optimization (MCO) problem. The number of competing objectives typically range from a few up to about ten, depending on the tumor location. Commonly, the different criteria are combined using a weighted sum, where each weight determines the relative importance of that criterion. For a convex optimization problem, optimization attempts to identify an accurate approximation of a complete set of Pareto optimal radiotherapy plans. In this context, a Pareto optimal plan refers to radiotherapy plans where no criterion can be improved without worsening another.

Finding acceptable weights to develop a Pareto optimal radiotherapy plan is often a manual and tedious process of trial-and-error, especially because evaluating a single choice of parameters requires solving a full optimization problem for the radiotherapy treatment. Solving a full optimization problem for a single plan may take from a few seconds up to an hour to calculate and evaluate the parameter combination, depending on the application. Additionally, even if a set of Pareto optimal plans is developed for a particular radiotherapy treatment, significant time and efforts may be needed to identify, select, and refine the plan which is most suitable for implementation.

### OVERVIEW

Various embodiments, methods, systems, and computer-readable mediums are provided for the evaluation and optimization of radiotherapy plans, including exploration or navigation of the results of solving a radiotherapy problem (e.g., a radiotherapy problem that is adjustable via specific parameterized criteria and that is solved using an optimization method). This technique can be used to evaluate the results of an optimization method which has produced a plurality of possible solutions. Such evaluation then can be used to identify a particular parameter choice or combination of parameters that results in a satisfactory radiotherapy plan.

As discussed herein, a radiotherapy problem may be expressed as a multicriteria optimization (MCO) problem, where the parameters correspond to clinical preferences. The plurality of solutions generated for such a radiotherapy problem may constitute a plurality of Pareto optimal plans. A Pareto optimal plan, as used herein, refers to a plan which is optimized such that no criterion can be improved without worsening another. The set of all Pareto optimal plans constitutes the Pareto surface (also known as the Pareto frontier).

The following provides an expanded approach for radiotherapy treatment planning optimization using a specialized technique to navigate a Pareto surface of solutions to the MCO problem. In an example, the Pareto surface (which is path-connected) may be explored starting from a Pareto-optimal initial point, using a submanifold that is lower-dimensional in weight space. The solutions to the MCO problem then may be estimated based on derivatives of first-order optimality conditions.

In various examples, operations for such radiotherapy treatment planning may include a process for: obtaining a plurality of solutions, defined in a manifold space, of a radiotherapy problem for providing radiotherapy treatment, exploring the plurality of solutions to identify an additional solution in a submanifold space, and generating treatment plan parameters based on the additional solution, with the treatment plan parameters provided to be used in a treatment plan for delivery of the radiotherapy treatment via a radiotherapy machine. The radiotherapy problem is a multicriteria optimization problem, and each of the plurality of solutions has a plurality of weights used to adjust a plurality of criteria for the multicriteria optimization problem.

In an example, the identification of the additional solution and the exploration of the plurality of solutions based on: (a) establishing a submanifold space from a manifold space representing the plurality of solutions in fewer dimensions than the plurality of weights; (b) producing additional sets of weights in the submanifold space based on derivatives of first-order optimality conditions of the radiotherapy problem, with such derivatives determined with respect to the plurality of weights; and (c) performing navigation in the submanifold space to arrive at the additional solution, with this additional solution corresponding to one of the additional sets of weights. For instance, the navigation may be performed in the submanifold space using a one-dimensional path. Likewise, in an example, the navigation of the one-dimensional path may be performed starting from a Pareto-optimal initial point, as the navigation reduces to finding the one-dimensional path by solving an ordinary differential equation based on a directional derivative.

In a further example, the identifying of the additional solution solves a boundary value problem, with an initial point and a final point being provided for the navigation of the one-dimensional path, as the navigation interpolates between the initial point and the final point. Also in a further example, the identifying of the additional solution solves an initial value problem, and an initial point is provided for the navigation of the one-dimensional path, as the navigation operates until meeting a predetermined stopping condition. Such a predetermined stopping condition may be at least one of: time, distance, or a maximum acceptable deterioration of a clinical metric (e.g., with deterioration determined using a simultaneous localization and mapping (SLAM) method to improve a first clinical metric while minimizing deterioration of a second clinical metric).

In further examples, the additional sets of weights in the submanifold space correspond to a level-set of the optimality conditions of the radiotherapy problem. Also in further examples, the derivatives are provided by automated differentiation. Also in further examples, at least a portion of the manifold space has a non-differentiable portion. In such a non-differentiable scenario, a least-squares method may be used to smoothly approximate the non-differentiable portion of the manifold space; or, a barrier formulation may be used to convert the constrained problem into an unconstrained problem, by including constraints as terms in an objective function that associates violations of the constraints with penalties.

In further example, the plurality of criteria for the radiotherapy problem correspond to clinical preferences. For instance, parameters of the plurality of criteria may concern a particular anatomical area to receive the radiotherapy treatment from the radiotherapy machine. Likewise, the plurality of criteria may include definitions of one or more organ at risk areas and one or more target areas.

In further examples, a representation may be generated and output for potential solutions, the additional solution, or other aspects of solution exploration for the radiotherapy problem. Graphical representations may include a display provided from a graphical user interface that has functionality to configure the treatment plan, and a display of information within the graphical user interface that provides information associated with the additional solution. This graphical user interface or other user interfaces may also receive a user or automated selection of the additional solution. In a still further example, the selected additional solution provides a warm start to identify a final solution used for the treatment plan, and additional optimization(s) are received to the selected additional solution before the treatment plan data is generated from the final solution.

The treatment plan parameters for the radiotherapy treatment may comprise a set of treatment delivery parameters corresponding to capabilities of the radiotherapy treatment machine. In an example, the treatment plan is used to provide the radiotherapy treatment with a Gamma knife, and the set of treatment delivery parameters comprises a set of isocenters used for delivery of the radiotherapy treatment. For instance, the set of treatment delivery parameters further comprises timing for delivery of the radiotherapy treatment and a collimator sequence for the delivery of the radiotherapy treatment with the Gamma knife. As another example, the treatment plan is used to provide the radiotherapy treatment with a Volumetric-modulated arc therapy (VMAT) or Intensity modulated radiation therapy (IMRT), e.g., using a Linac radiotherapy machine, and the set of treatment delivery parameters comprises one or more of: a set of arc control points for one or more arcs, fluence fields, gantry speed, and dose rate along the one or more arcs.

In further examples, the operations may be followed by operations that cause or effect the delivery of the radiotherapy treatment using a plurality of radiotherapy beams from the radiotherapy treatment machine, based on the treatment plan parameters or other data generated or identified as discussed herein.

The above overview is intended to provide an overview of subject matter of the present patent application. It is not intended to provide an exclusive or exhaustive explanation of the inventive subject matter. The detailed description is included to provide further information about the present patent application.

### BRIEF DESCRIPTION OF THE DRAWINGS

In the drawings, which are not necessarily drawn to scale, like numerals describe substantially similar components throughout the several views. Like numerals having different letter suffixes represent different instances of substantially similar components. The drawings illustrate generally, by way of example but not by way of limitation, various embodiments discussed in the present document.

FIG. 1 illustrates a radiotherapy system, according to some examples.

FIG. 2A illustrates a radiotherapy system having output configured to provide a therapy beam, according to some examples.

FIG. 2B illustrates a system including a combined radiation therapy system and an imaging system, such as a cone beam computed tomography (CBCT) imaging system, according to some examples.

FIG. 3 illustrates a partially cut-away view of a system including a combined radiation therapy system and an imaging system, such as a nuclear magnetic resonance (MR) imaging (MRI) system, according to some examples.

FIG. 4 illustrates an example of a Leksell Gamma Knife radiotherapy device, according to some examples.

FIG. 5 illustrates a radiotherapy treatment planning workflow, according to some examples.

FIG. 6 illustrates a further workflow for identification and exploration of additional Pareto-optimal solutions, according to some examples.

FIG. 7 illustrates a representation of exploration in a Pareto surface, according to some examples.

FIGS. 8A-8D illustrate a representation of a Pareto surface and submanifolds, according to some examples.

FIG. 9 illustrates a flowchart for a method of radiotherapy treatment planning, according to some examples.

FIG. 10 illustrates an exemplary block diagram of a machine on which one or more of the methods as discussed herein can be implemented.

### DETAILED DESCRIPTION

In the following detailed description, reference is made to the accompanying drawings which form a part hereof, and which is shown by way of illustration-specific embodiments in which the present disclosure may be practiced. These embodiments, which are also referred to herein as “examples,” are described in sufficient detail to enable those skilled in the art to practice the disclosure, and it is to be understood that the embodiments may be combined, or that other embodiments may be utilized and that structural, logical and electrical changes may be made without departing from the scope of the present disclosure. The following detailed description is, therefore, not to be taken in a limiting sense, and the scope of the present disclosure is defined by the appended claims and their equivalents.

The following discusses various implementations of planning and optimization techniques usable in radiotherapy or radiosurgery applications. Such techniques may be used to find and evaluate new solutions to a radiotherapy problem, which may correspond to different parameter choices and values (weights) for such parameterized criteria. Based on the exploration, selection, and refinement of such parameters and parameter values, treatment plan data and a unique treatment plan for implementation with a radiotherapy treatment machine may be generated.

In the examples discussed herein, a plurality of solutions for a radiotherapy optimization problem may be evaluated during a process for “surfing” a Pareto surface of solutions. Starting from a Pareto optimal initial point, the following techniques provide an exploration of the Pareto surface-which is path-connected-using a submanifold that is lower-dimensional in weight space. Additional solutions to a multi-criteria radiotherapy optimization problem expressed by the submanifold may be estimated based on derivatives of the first-order optimality conditions. The first-order optimality conditions are necessary conditions for a point to be optimal. For an unconstrained optimization problem, stationarity (i.e., that the gradient of the objective function is zero) is a first-order optimality condition. For a constrained optimization problem, compliance with Karush-Kuhn-Tucker (KKT) conditions are a first-order optimality condition.

The presently disclosed exploration and generation of radiotherapy problem solutions offers significant technical and clinical benefits. The technical benefits include reduced computing processing times to generate new radiotherapy treatment plans, enhanced computation solutions for radiotherapy treatment plan optimization problems, and accompanying improvements in processing, memory, and network resources used to generate or perform radiotherapy treatments. The related clinical benefits include potentially improved plan quality and reduced time for treatment.

It is extremely challenging, if at all possible, to capture a treatment planner's preferences and requirements in a single mathematical expression. Even if such a model could be obtained, it would almost certainly be very difficult to optimize. Instead, the following uses a set of well-behaved (e.g. convex) surrogate functions as building blocks which, when combined appropriately, result in treatment plans that meet the actual preferences and requirements of the treatment planner. To ensure that such plans exist, the surrogate functions are often chosen such that they correlate with particular clinical considerations and, together, span the relevant range of treatment plans. However, because of the mismatch between the actual clinical considerations and the surrogate functions, and because the true clinical preferences are generally unknown, neither is it known how to weight the surrogate functions to find a clinically satisfactory plan.

It is therefore necessary to evaluate different weight settings. This is in general a tedious, time consuming process since an optimization must be performed at each step. Consequently, it is unlikely that more than a few attempts will be made with the risk that the finally chosen plan is inferior to what could have been achieved. Indeed, multiple studies have found that treatment plan quality is strongly dependent on both the time-commitment and the experience level of the individual treatment planner.

Generating the collection of a large number of plans (i.e. the approximation of the Pareto surface) in the same time as a single optimization and providing tools to navigate on this potentially high-dimensional surface, increases the likelihood of finding a much better plan for the patient in a shorter time than an optimization process involving manual intervention. Further, an approximation of the Pareto surface provides the treatment planner with an overview of the achievable tradeoffs, which makes it easier to identify a promising area to explore further as well as making it easier to know when to terminate the search for better plans. Both these aspects lead to shorter planning times and potentially higher plan quality on average.

The technique used for selection of the clinical plan from the Pareto approximation is also important. Discrete selection of one of the precalculated plans requires a very dense representation that is not feasible to generate in practice. A much coarser representation is sufficient if continuous interpolation between a discrete set of solutions is allowed. Continuous interpolation of this form is often called navigation. Plans optimized based on a weighted sum of objective (surrogate) functions can be proven to be Pareto optimal with respect to those objectives but not necessarily with respect to the actual clinical considerations. The navigation tools provided by the following techniques enables the clinician to explore and prioritize plans according to the actual clinical considerations, thereby identifying the best plan faster and more reliably.

As is explained in the sections below, the following techniques may be used to represent and explore a large set of treatment plans, such as a Pareto surface of Pareto-optimal treatment plans, allowing the discovery of new treatment parameter weights that can be identified and selected for use. Prior approaches for representing and exploring a representative set of treatment plans with conventional processing techniques often resulted in significant computation time and delays. For example, some prior approaches involve pre-calculating a representative set of Pareto optimal treatment plans, and choosing a most suitable plan among those. The number of representative plans needed to span the Pareto surface for a Gamma Knife treatment is typically on the order of hundreds or thousands of plans, which means that a very long computation time (typically an over-night run) would be required to generate a full representation of possible solutions. Some prior approaches also attempted to select and combine weights from different solutions, such as with use of an interpolation scheme. However, evaluation and exploration of such solutions is quite computationally intensive, requiring immense amounts of sampling. In contrast, the following discusses techniques for efficient Pareto navigation, and interactive steps which are taken to identify a satisfactory tradeoff given a starting set of Pareto optimal plans.

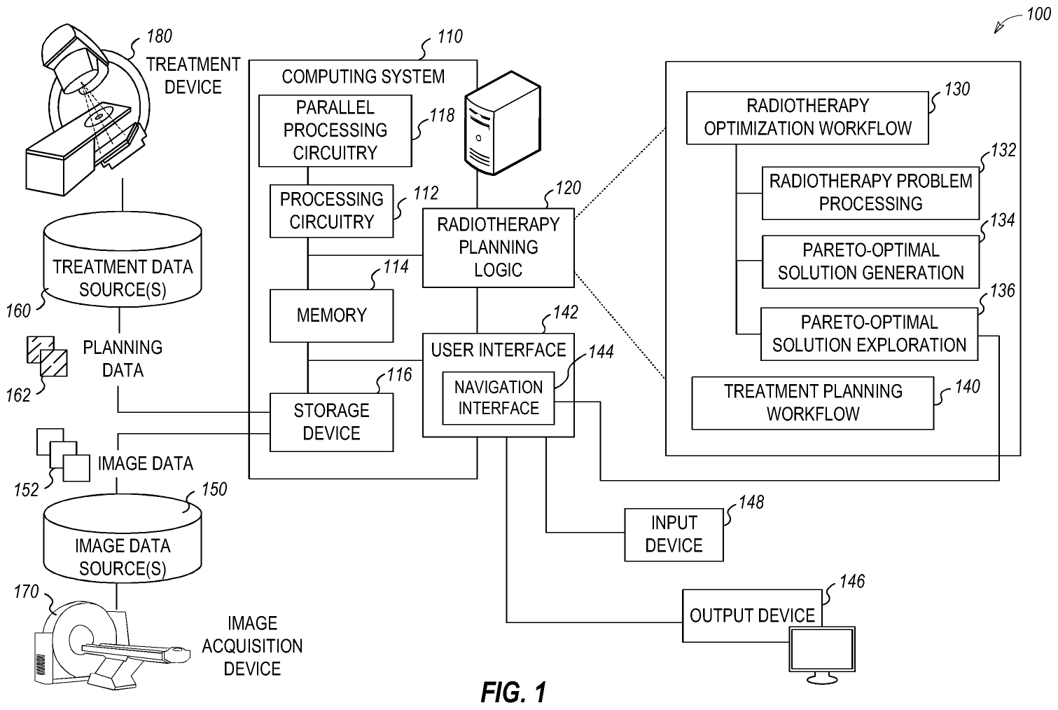

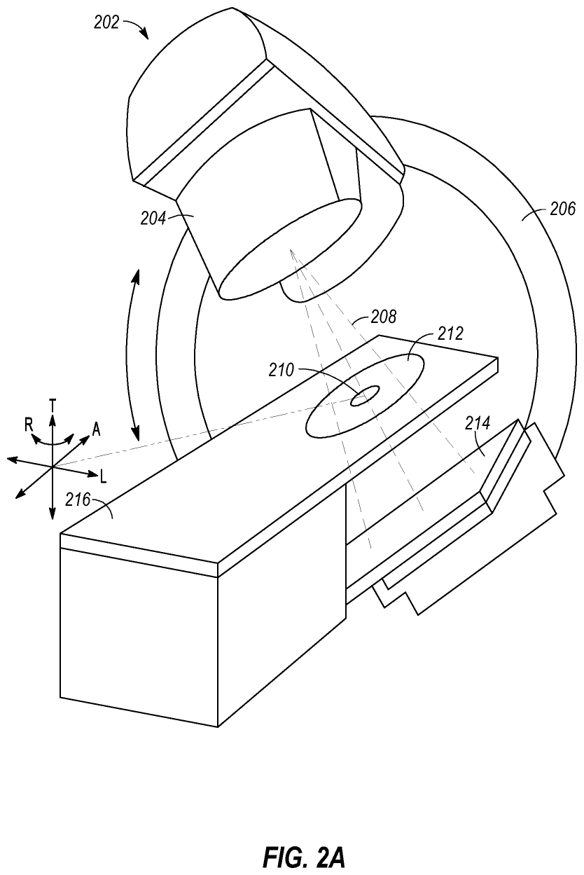

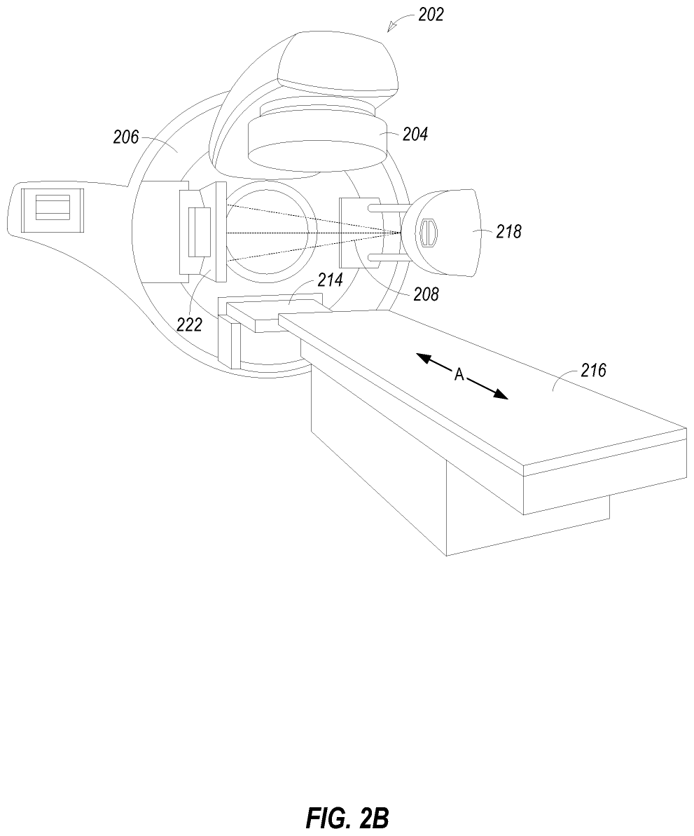

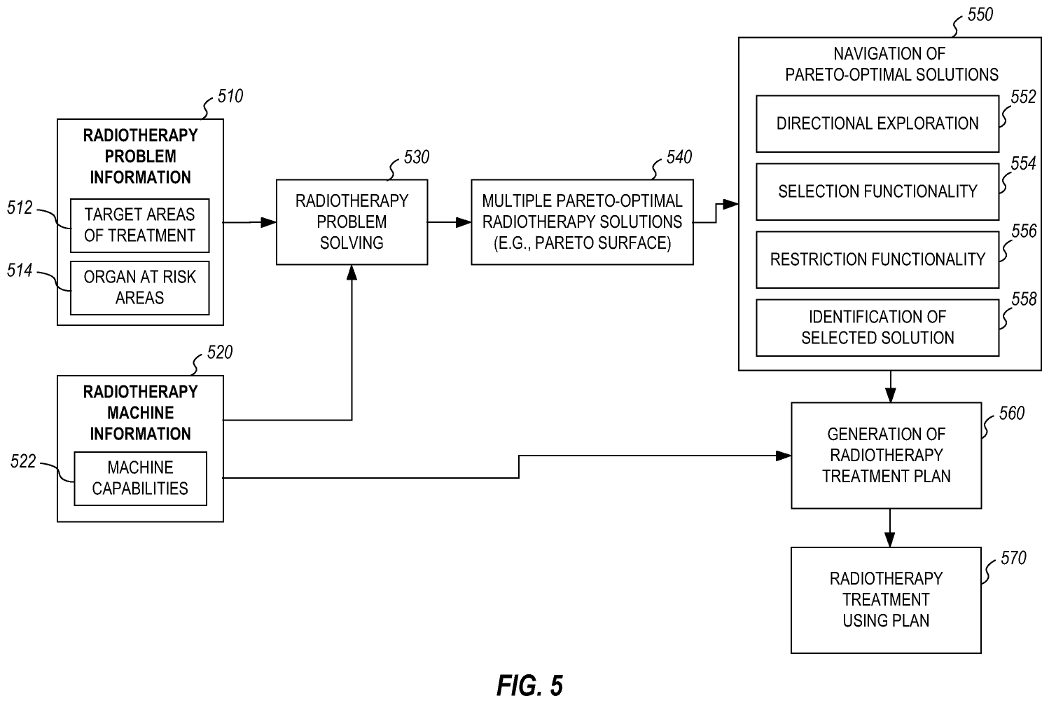

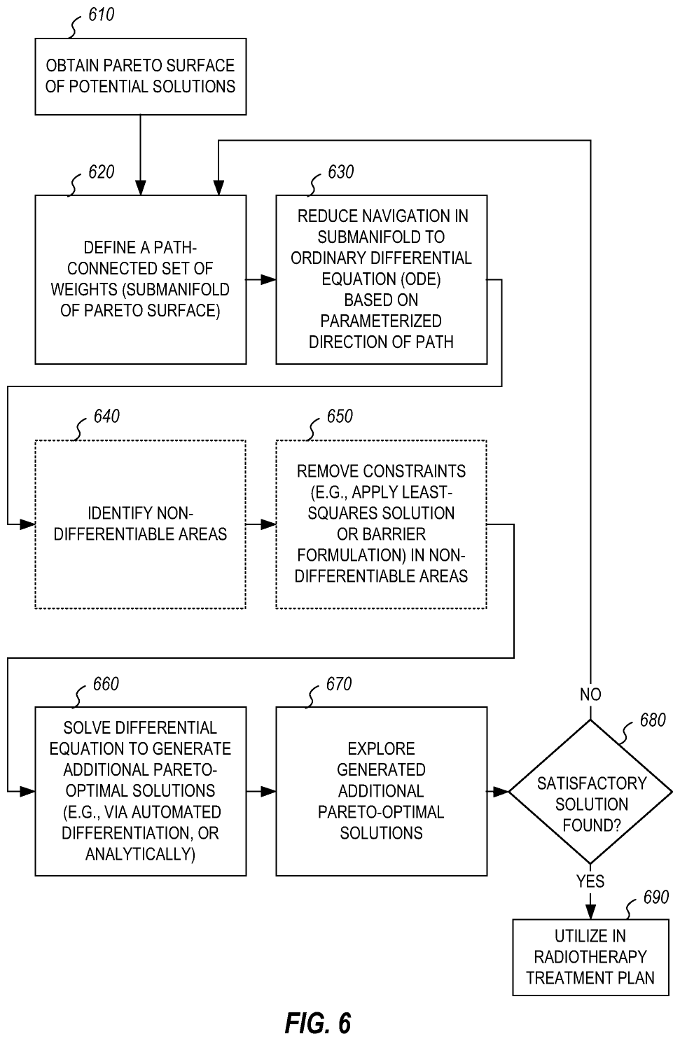

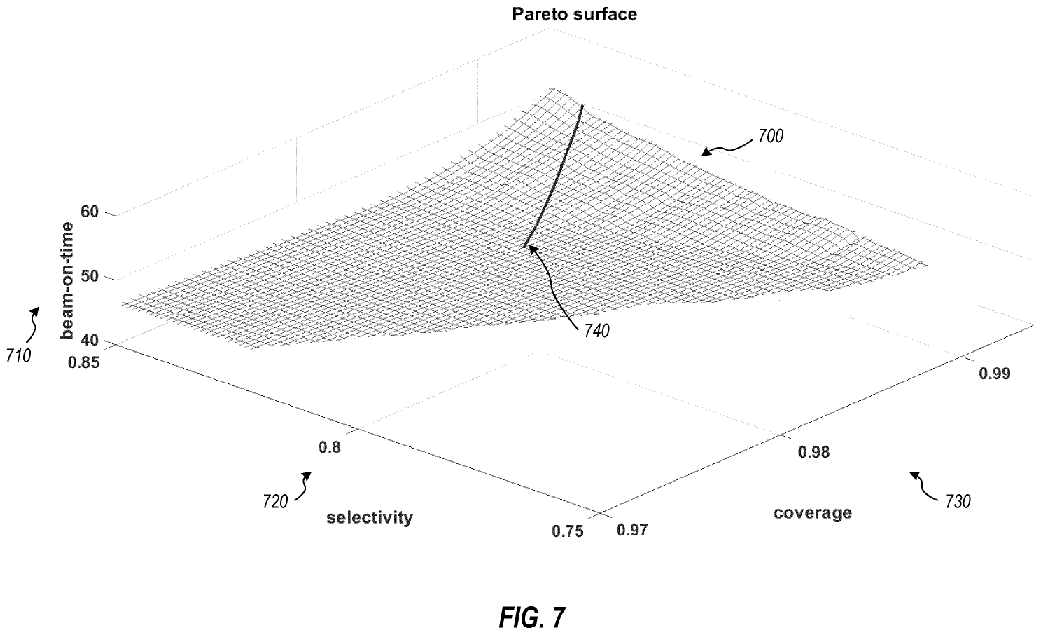

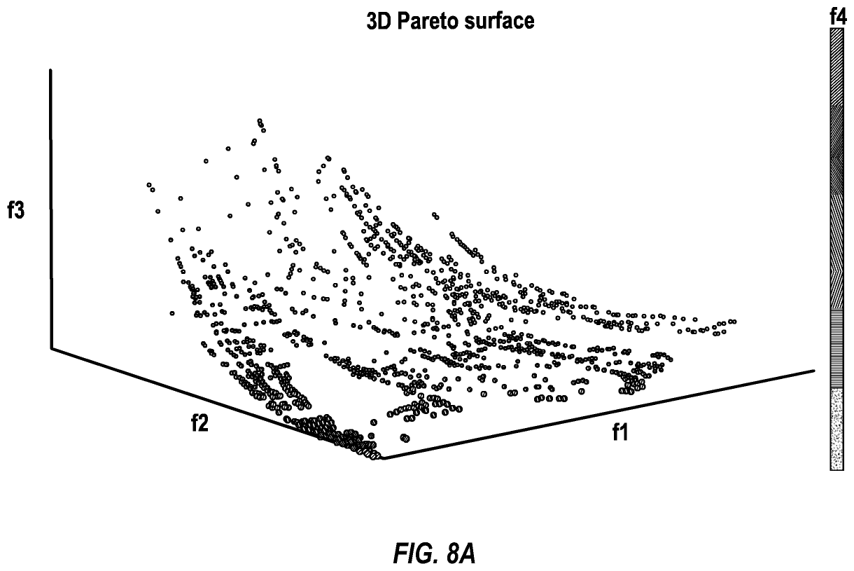

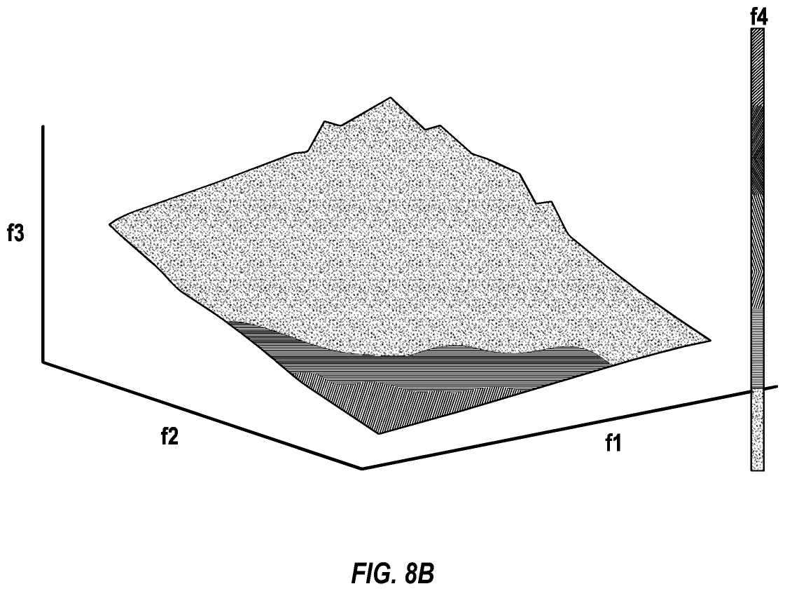

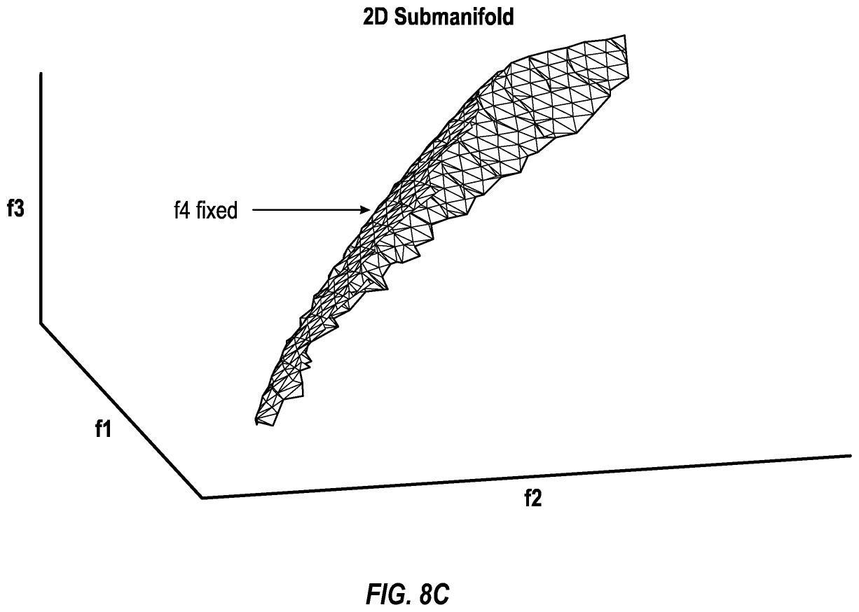

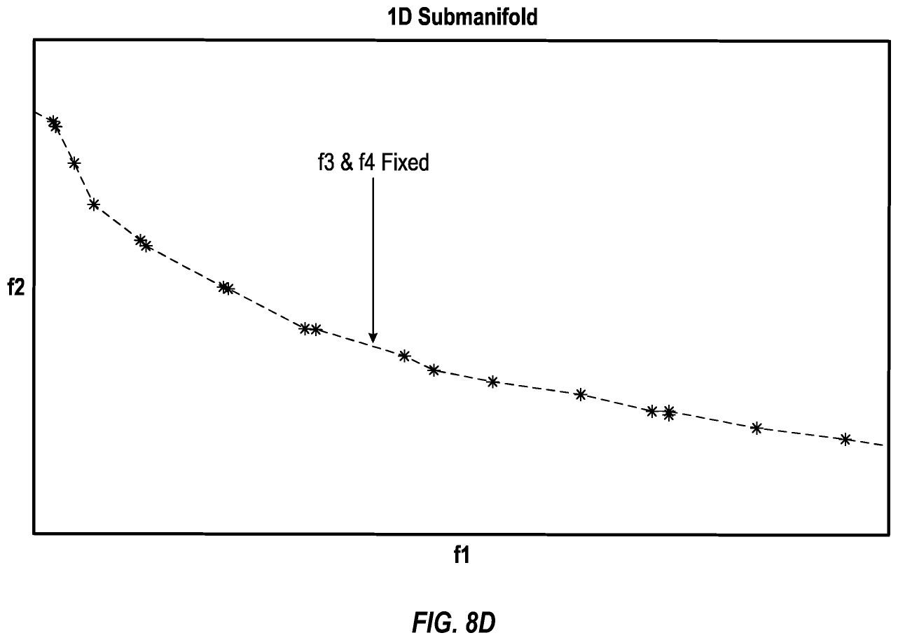

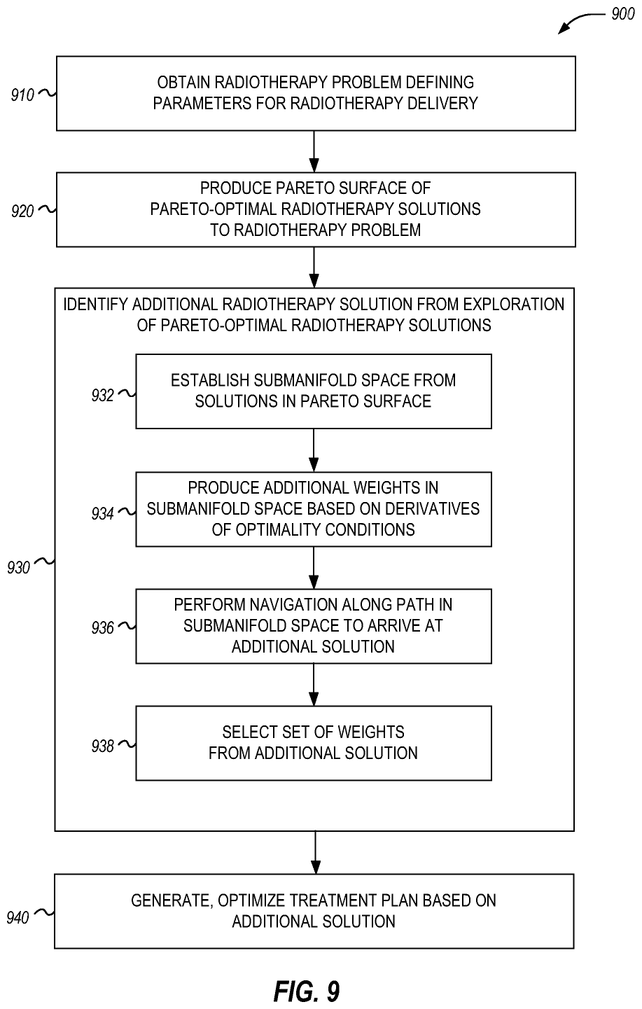

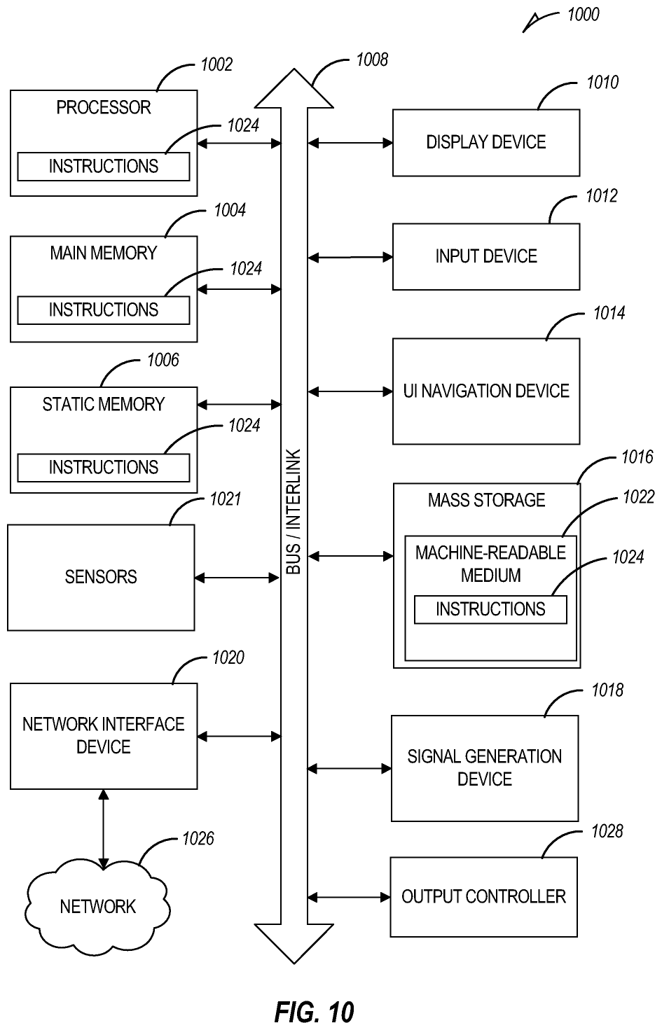

The following paragraphs provide an overview of example radiotherapy system implementations and treatment use cases (with reference to FIGS. 2A, 2B, 3 and 4), including with the use of computing systems and hardware implementations (with reference to FIGS. 1 and 10). The following then continues with a discussion of example treatment plan exploration and development workflows (with reference to FIGS. 5 and 6) including ways in which a representation of Pareto surface can be explored (with reference to the representation of a Pareto surface in FIGS. 7 and 8). Finally, a discussion of radiotherapy treatment planning (with reference to FIG. 9) is provided, which illustrates an end-to-end method of generating an optimized treatment plan.

FIG. 1 illustrates an exemplary radiotherapy system 100 adapted to perform radiotherapy plan processing operations using one or more of the approaches discussed herein. These radiotherapy plan processing operations are performed to enable the radiotherapy system 100 to provide radiation therapy to a patient based on specific aspects of captured medical imaging data and therapy dose calculations or radiotherapy machine configuration parameters. Specifically, the following processing operations may be implemented as part of the radiotherapy planning logic 120 for exploring and developing weights of parameters for a radiotherapy treatment plan. It will be understood, however, that many variations and use cases of the following planning logic 120 and optimization operations may be provided, such as in response to data verification, visualization, and other medical evaluative and diagnostic operations.

The radiotherapy system 100 includes a radiotherapy processing computing system 110 which hosts radiotherapy planning logic 120. The radiotherapy processing computing system 110 may be connected to a network (not shown), and such network may be connected to the Internet. For instance, a network can connect the radiotherapy processing computing system 110 with one or more private and/or public medical information sources (e.g., a radiology information system (RIS), a medical record system (e.g., an electronic medical record (EMR)/electronic health record (EHR) system), an oncology information system (OIS)), one or more image data sources 150, an image acquisition device 170 (e.g., an imaging modality), a treatment device 180 (e.g., a radiation therapy device), and a treatment data source 160.

As an example, the radiotherapy processing computing system 110 can be configured to receive a treatment goal of a subject (e.g., from one or more MR images) and generate a radiotherapy treatment plan by executing instructions or data from the radiotherapy planning logic 120, as part of operations to generate treatment plans to be used by the treatment device 180 and/or for output on device 146. In an embodiment, the radiotherapy planning logic 120 generates solutions to an optimization problem, with one or more of solutions then used to generate the radiotherapy treatment plan. The radiotherapy planning logic 120 solves the radiotherapy optimization problem by estimating optimization variables of the received optimization problem. Then, the optimization problem is solved using an optimization problem solver, producing a plurality of possible solutions (e.g., Pareto-optimal solutions). The possible solutions to the radiotherapy optimization problem are then further explored and refined using the techniques discussed herein.

Example optimization problem solvers include, e.g., a simplex method, an interior point method, a Newton method, a quasi-Newton method, a Gauss-Newton method, a Levenberg-Marquardt method, a linear least-squares method, a gradient descent method, a projected gradient method, a conjugate gradient method, an augmented Lagrangian method, a Nelder-Mead method, a branch and bound method, a cutting plane method, simulated annealing, and/or sequential quadratic programming. In further examples, optimization problem solvers may include the use of an alternating direction method of multipliers (ADMM) applied on parallel processing circuitry. Another class of optimization solvers employs a machine learning model that predicts the solution or some intermediate result of the computation, and may be combined or interleaved with steps performed according to an optimization method not based on machine learning.

A generic radiotherapy treatment plan optimization problem can be defined as Equation 1:

$${minimize}_{x ∈ X} f ( x ) \qquad ( Equation 1 )$$

$$subject to x ∈ Ω$$

where ƒ:X→ [symbol] is the objective function, x∈X is the decision variables (also referred to as optimization variables) and Ω⊆X is the set of feasible variables. In general, the function ƒ can be nonlinear and the set Ω non-convex. The optimization problems are typically solved using some form of iterative scheme. For example, in case ƒ is smooth and convex, and Ω is convex, then the projected gradient scheme could be used to solve equation (1) and reads as follows:

$${x}_{n + 1} = {proj}_{Ω} ( {x}_{n} - η ∇ f ( {x}_{n} ) ) \qquad ( Equation 2 )$$

where proj_{Ω}: X→X is the projection onto Ω, n∈ [symbol] is a stepsize and ∇ƒ: X→X the gradient. Such algorithms are typically provably convergent (e.g., given enough time (and correct parameter choices), the algorithm will converge to a minimizer). Some of these algorithms may require hundreds if not thousands of iterations in order to achieve approximate convergence. Since each step may be computationally expensive, this may imply runtimes of minutes or even hours. However, use of the techniques discussed herein enables exploration of such solutions, and the creation of additional parameter values based on these solutions.

The radiotherapy processing computing system 110 may include processing circuitry 112, memory 114, a storage device 116, and other hardware and software-operable features such as a user interface 142, a communication interface (not shown), and the like. The storage device 116 may store transitory or non-transitory computer-executable instructions, such as an operating system, radiation therapy treatment plans, training data, software programs (e.g., image processing software, image or anatomical visualization software, artificial intelligence (AI) or ML implementations and algorithms such as provided by deep learning models, ML models, and neural networks (NNs), etc.), and any other computer-executable instructions to be executed by the processing circuitry 112.

In an example, the processing circuitry 112 may include a processing device, such as one or more general-purpose processing devices such as a microprocessor, a central processing unit (CPU), a graphics processing unit (GPU), an accelerated processing unit (APU), or the like. More particularly, the processing circuitry 112 may be a complex instruction set computing (CISC) microprocessor, a reduced instruction set computing (RISC) microprocessor, a very long instruction Word (VLIW) microprocessor, a processor implementing other instruction sets, or processors implementing a combination of instruction sets. The processing circuitry 112 may also be implemented by one or more special-purpose processing devices such as an application-specific integrated circuit (ASIC), a field programmable gate array (FPGA), a digital signal processor (DSP), a System on a Chip (SoC), or the like.

As would be appreciated by those skilled in the art, in some examples, the processing circuitry 112 may be a special-purpose processor rather than a general-purpose processor. The processing circuitry 112 may include one or more known processing devices, such as a microprocessor from the Pentium™, Core™, Xeon™, or Itanium® family manufactured by Intel™, the Turion™, Athlon™, Sempron™, Opteron™, FX™, Phenom™ family manufactured by AMD™, or any of various processors manufactured by Sun Microsystems. The processing circuitry 112 may also include graphical processing units such as a GPU from the GeForce®, Quadro®, Tesla® family manufactured by Nvidia™, GMA, Iris™ family manufactured by Intel™, or the Radeon™ family manufactured by AMD™. The processing circuitry 112 may also include accelerated processing units such as the Xeon Phi™ family manufactured by Intel™. The disclosed embodiments are not limited to any type of processor(s) otherwise configured to meet the computing demands of identifying, analyzing, maintaining, generating, and/or providing large amounts of data or manipulating such data to perform the methods disclosed herein. In addition, the term “processor” may include more than one physical (circuitry-based) or software-based processor (for example, a multi-core design or a plurality of processors each having a multi-core design).

Other implementations of the processing circuitry 112 may include processors or processing units arranged into a parallel processing configuration. For instance, a set of graphical processing units (e.g., GPUs from the GeForce®, Quadro®, Tesla® family manufactured by Nvidia™, GMA, Iris™ family manufactured by Intel™, or the Radeon™ family manufactured by AMD™) may be arranged to perform highly parallel or repetitive computing tasks simultaneously. Other specialized parallel processing units or hardware capable of performing multiple calculations simultaneously may also be deployed. GPUs may include a single GPU “device” or “system” which operates or orchestrates numerous (e.g., tens, hundreds, or thousands) of sub-processors; use of a GPU may include use of a single GPU device or system which uses each of its numerous sub-processors to process a respective set of parameters.

The processing circuitry 112 can execute sequences of transitory or non-transitory computer program instructions, stored in memory 114, and accessed from the storage device 116, to perform various operations, processes, and methods that will be explained in greater detail below. It should be understood that any component in system 100 may be implemented separately and operate as an independent device and may be coupled to any other component in system 100 to perform the techniques described in this disclosure.

The memory 114 may comprise read-only memory (ROM), a phase-change random access memory (PRAM), a static random access memory (SRAM), a flash memory, a random access memory (RAM), a dynamic random access memory (DRAM) such as synchronous DRAM (SDRAM), an electrically erasable programmable read-only memory (EEPROM), a static memory (e.g., flash memory, flash disk, static random access memory) as well as other types of random access memories, a cache, a register, a compact disc read-only memory (CD-ROM), a digital versatile disc (DVD) or other optical storage, a cassette tape, other magnetic storage device, or any other non-transitory medium that may be used to store information including images, training data, one or more ML model(s) or technique(s) parameters, data, or transitory or non-transitory computer executable instructions (e.g., stored in any format) capable of being accessed by the processing circuitry 112, or any other type of computer device. For instance, the computer program instructions can be accessed by the processing circuitry 112, read from the ROM, or any other suitable memory location, and loaded into the RAM for execution by the processing circuitry 112.

The storage device 116 may constitute a drive unit that includes a transitory or non-transitory machine-readable medium on which is stored one or more sets of transitory or non-transitory instructions and data structures (e.g., software) embodying or utilized by any one or more of the methodologies or functions described herein (including, in various examples, the radiotherapy planning logic 120 and the user interface 142). The instructions may also reside, completely or at least partially, within the memory 114 and/or within the processing circuitry 112 during execution thereof by the radiotherapy processing computing system 110, with the memory 114 and the processing circuitry 112 also constituting transitory or non-transitory machine-readable media.

The memory 114 and the storage device 116 may constitute a non-transitory computer-readable medium. For example, the memory 114 and the storage device 116 may store or load transitory or non-transitory instructions for one or more software applications on the computer-readable medium. Software applications stored or loaded with the memory 114 and the storage device 116 may include, for example, an operating system for common computer systems as well as for software-controlled devices. The radiotherapy processing computing system 110 may also operate a variety of software programs comprising software code for implementing the radiotherapy planning logic 120 and the user interface 142. Further, the memory 114 and the storage device 116 may store or load an entire software application, part of a software application, or code or data that is associated with a software application, which is executable by the processing circuitry 112. In a further example, the memory 114 and the storage device 116 may store, load, and manipulate one or more radiation therapy treatment plans, imaging data, segmentation data, treatment visualizations, histograms or measurements, one or more AI model data (e.g., weights and parameters of one or more ML model(s)), training data, labels and mapping data, and the like. It is contemplated that software programs may be stored not only on the storage device 116 and the memory 114 but also on a removable computer medium, such as a hard drive, a computer disk, a CD-ROM, a DVD, a Blu-Ray DVD, USB flash drive, a SD card, a memory stick, or any other suitable medium; such software programs may also be communicated or received over a network.

Although not depicted, the radiotherapy processing computing system 110 may include a communication interface, network interface card, and communications circuitry. An example communication interface may include, for example, a network adaptor, a cable connector, a serial connector, a USB connector, a parallel connector, a high-speed data transmission adaptor (e.g., such as fiber, USB 3.0, thunderbolt, and the like), a wireless network adaptor (e.g., such as an IEEE 802.11/Wi-Fi adapter), a telecommunication adapter (e.g., to communicate with 3G, 4G/LTE, and 5G networks and the like), and the like. Such a communication interface may include one or more digital and/or analog communication devices that permit a machine to communicate with other machines and devices, such as remotely located components, via a network. The network may provide the functionality of a local area network (LAN), a wireless network, a cloud computing environment (e.g., software as a service, platform as a service, infrastructure as a service, etc.), a client-server, a wide area network (WAN), and the like. For example, the network may be a LAN or a WAN that may include other systems (including additional image processing computing systems or image-based components associated with medical imaging or radiotherapy operations).

In an example, the radiotherapy processing computing system 110 may obtain image data 152 from the image data source 150 (e.g., MR images) for hosting on the storage device 116 and the memory 114. In yet another example, the software programs may substitute functions of the patient images such as signed distance functions or processed versions of the images that emphasize some aspect of the image information. The radiotherapy processing computing system 110 may obtain or communicate image data 152 from or to image data source 150. In further examples, the treatment data source 160 receives or updates the planning data 162 as a result of a treatment plan generated by the radiotherapy planning logic 120. The image data source 150 may also provide or host the imaging data for use in the radiotherapy planning logic 120.

In an example, computing system 110 may communicate with treatment data source(s) 160, input device 148, and other data sources to generate optimized parameters and weights for a plurality of radiotherapy treatment plan optimization problems. Such optimization variables and parameters are then evaluated and refined to identify a specific solution to the radiotherapy problem. It will be understood that the results of solution generation and solution exploration may approximate an ultimately implemented solution, but may provide a warm start to identify the true solution. Thus, multiple iterations of generation, identification, exploration, and refinement may be involved before use of a solution in a radiotherapy treatment.

The processing circuitry 112 may be communicatively coupled to the memory 114 and the storage device 116, and the processing circuitry 112 may be configured to execute computer-executable instructions stored thereon from either the memory 114 or the storage device 116. Particularly, radiotherapy planning logic 120 receives an optimization problem that is derived from parameters for radiotherapy treatment. The processing circuitry 112 may utilize software programs or implementations to explore and develop parameters for delivering a radiotherapy dose to a patient, as part of developing an optimized solution to a radiotherapy problem as discussed herein. Further, such software programs or implementations may utilize the radiotherapy planning logic 120 to produce new or updated treatment plan parameters for deployment to the treatment data source 160 and/or presentation on output device 146, using the techniques further discussed herein. The processing circuitry 112 may subsequently then transmit the new or updated treatment plan details via a communication interface and the network to the treatment device 180, where the radiation therapy plan will be used to treat a patient with radiation via the treatment device 180, consistent with results of the radiotherapy planning logic 120 (e.g., according to the processes discussed below).

In an example, the image data 152 used for defining a radiotherapy problem or indicating the anatomical areas of the patient may include one or more MRI image (e.g., 2D MRI, 3D MRI, 2D streaming MRI, 4D MRI, 4D volumetric MRI, 4D cine MRI, etc.), functional MRI images (e.g., fMRI, DCE-MRI, diffusion MRI), Computed Tomography (CT) images (e.g., 2D CT, 2D Cone beam CT, 3D CT, 3D CBCT, 4D CT, 4DCBCT), ultrasound images (e.g., 2D ultrasound, 3D ultrasound, 4D ultrasound), Positron Emission Tomography (PET) images, X-ray images, fluoroscopic images, radiotherapy portal images, Single-Photo Emission Computed Tomography (SPECT) images, computer-generated synthetic images (e.g., pseudo-CT images) and the like. Further, the image data 152 may also include or be associated with medical image processing data (for example, training images, ground truth images, contoured images, and dose images). In other examples, an equivalent representation of an anatomical area may be represented in non-image formats (e.g., coordinates, mappings, etc.).

In an example, the image data 152 may be received from the image acquisition device 170 and stored in one or more of the image data sources 150 (e.g., a Picture Archiving and Communication System (PACS), a Vendor Neutral Archive (VNA), a medical record or information system, a data warehouse, etc.). Accordingly, the image acquisition device 170 may comprise an MRI imaging device, a CT imaging device, a PET imaging device, an ultrasound imaging device, a fluoroscopic device, a SPECT imaging device, an integrated Linear Accelerator and MRI imaging device, CBCT imaging device, or other medical imaging devices for obtaining the medical images of the patient. The image data 152 may be received and stored in any type of data or any type of format (e.g., in a Digital Imaging and Communications in Medicine (DICOM) format) that the image acquisition device 170 and the radiotherapy processing computing system 110 may use to perform operations consistent with the disclosed embodiments. Further, in some examples, the models discussed herein may be trained to process the original image data format or a derivation thereof.

In an example, the image acquisition device 170 may be integrated with the treatment device 180 as a single apparatus (e.g., an MRI device combined with a linear accelerator, also referred to as an “MRI-Linac”). Such an MRI-Linac can be used, for example, to determine a location of a target organ or a target tumor in the patient so as to direct radiation therapy accurately according to the radiation therapy treatment plan to a predetermined target. For instance, a radiation therapy treatment plan may provide information about a particular radiation dose to be applied to each patient. The radiation therapy treatment plan may also include other radiotherapy information, including control points of a radiotherapy treatment device, such as couch position, beam intensity, beam angles, dose-histogram-volume information, the number of radiation beams to be used during therapy, the dose per beam, and the like.

The radiotherapy processing computing system 110 may communicate with an external database through a network to send/receive a plurality of various types of data related to image processing and radiotherapy operations. For example, an external database may include machine data (including device constraints) that provides information associated with the treatment device 180, the image acquisition device 170, or other machines relevant to radiotherapy or medical procedures. Machine data information (e.g., control points) may include radiation beam size, arc placement, beam on and off time duration, machine parameters, segments, multi-leaf collimator (MLC) configuration, gantry speed, MRI pulse sequence, and the like. The external database may be a storage device and may be equipped with appropriate database administration software programs. Further, such databases or data sources may include a plurality of devices or systems located either in a central or a distributed manner.

The radiotherapy processing computing system 110 can collect and obtain data, and communicate with other systems, via a network using one or more communication interfaces, which are communicatively coupled to the processing circuitry 112 and the memory 114. For instance, a communication interface may provide communication connections between the radiotherapy processing computing system 110 and radiotherapy system components (e.g., permitting the exchange of data with external devices). For instance, the communication interface may, in some examples, have appropriate interfacing circuitry from an output device 146 or an input device 148 to connect to the user interface 142, which may be a hardware keyboard, a keypad, or a touch screen through which a user may input information into the radiotherapy system.

As an example, the output device 146 may include a display device that outputs a representation of the user interface 142 and one or more aspects, visualizations, or representations of the medical images, the treatment plans, solutions or solution spaces, and statuses of training, generation, verification, or implementation of such plans. The output device 146 may include one or more display screens that display medical images, interface information, treatment planning parameters (e.g., contours, dosages, beam angles, labels, maps, etc.), treatment plans, a target, localizing a target and/or tracking a target, or any related information to the user. In a specific example, the user interface 142 includes a solution navigation interface with allows manipulation and interaction with aspects of solution exploration 136 (such as with use of a graphical interface discussed below).

The input device 148 connected to the user interface 142 may be a keyboard, a keypad, a touch screen or any type of device that a user may use to the radiotherapy system 100. Alternatively, the output device 146, the input device 148, and features of the user interface 142 may be integrated into a single device such as a smartphone or tablet computer (e.g., Apple iPad®, Lenovo Thinkpad®, Samsung Galaxy®, etc.).

Furthermore, any and all components of the radiotherapy system may be implemented as a virtual machine (e.g., via VMWare, Hyper-V, and the like virtualization platforms) or independent devices. For instance, a virtual machine can be software that functions as hardware. Therefore, a virtual machine can include at least one or more virtual processors, one or more virtual memories, and one or more virtual communication interfaces that together function as hardware. For example, the radiotherapy processing computing system 110, the image data sources 150, or like components, may be implemented as a virtual machine or within a cloud-based virtualization environment.

The image acquisition device 170 can be configured to acquire one or more images of the patient's anatomy for a region of interest (e.g., a target organ, a target tumor or both). Each image, typically a 2D image or slice, can include one or more parameters (e.g., a 2D slice thickness, an orientation, and a location, etc.). In an example, the image acquisition device 170 can acquire a 2D slice in any orientation. For example, an orientation of the 2D slice can include a sagittal orientation, a coronal orientation, or an axial orientation. The processing circuitry 112 can adjust one or more parameters, such as the thickness and/or orientation of the 2D slice, to include the target organ and/or target tumor. In an example, 2D slices can be determined from information such as a 3D CBCT or CT or MRI volume. Such 2D slices can be acquired by the image acquisition device 170 in “near real time” while a patient is undergoing radiation therapy treatment (for example, when using the treatment device 180 (with “near real time” meaning acquiring the data in at least milliseconds or less)).

The radiotherapy planning logic 120 in the radiotherapy processing computing system 110 implements a radiotherapy optimization workflow 130 and treatment planning workflow 140. The radiotherapy optimization workflow 130 may implement optimization operations for identifying and developing parameters of radiotherapy plans, while the treatment planning workflow 140 may implement operations for implementing such parameters of the radiotherapy plan for specific use via a radiotherapy machine. In specific examples, the radiotherapy optimization workflow 130 performs radiotherapy problem processing 132 to obtain and identify an optimization problem, Pareto-optimal solution generation 134 to identify and output Pareto-optimal solutions to the multi-criteria optimization problems, and Pareto-optimal solution exploration 136 to explore such solutions in a reduced submanifold space, and generate additional solutions. More details of the radiotherapy optimization workflow 130 are provided below with reference to FIGS. 6 and 7, including with the use of various approaches for exploration and generation of unique radiotherapy problem solutions. Likewise, more details of the treatment planning workflow 140 are provided below with reference to FIGS. 5 and 9, which indicate how a unique problem solution may be produced and implemented into a treatment planning process.

FIG. 2A illustrates a radiation therapy device 202 that may include a radiation source, such as an X-ray source or a linear accelerator, a couch 216, an imaging detector 214, and a radiation therapy output 204. The radiation therapy device 202 may be configured to emit a radiation beam 208 to provide therapy to a patient. The radiation therapy output 204 can include one or more attenuators or collimators, such as an MLC. An MLC may be used for shaping, directing, or modulating an intensity of a radiation therapy beam to the specified target locus within the patient. The leaves of the MLC, for instance, can be automatically positioned to define an aperture approximating a tumor cross-section or projection, and cause modulation of the radiation therapy beam. For example, the leaves can include metallic plates, such as comprising tungsten, with a long axis of the plates oriented parallel to a beam direction and having ends oriented orthogonally to the beam direction. Further, a “state” of the MLC can be adjusted adaptively during a course of radiation therapy treatment, such as to establish a therapy beam that better approximates a shape or location of the tumor or other target locus.

Referring back to FIG. 2A, a patient can be positioned in a region 212 and supported by the treatment couch 216 to receive a radiation therapy dose, according to a radiation therapy treatment plan. The radiation therapy output 204 can be mounted or attached to a gantry 206 or other mechanical support. One or more chassis motors (not shown) may rotate the gantry 206 and the radiation therapy output 204 around couch 216 when the couch 216 is inserted into the treatment area. In an example, gantry 206 may be continuously rotatable around couch 216 when the couch 216 is inserted into the treatment area. In another example, gantry 206 may rotate to a predetermined position when the couch 216 is inserted into the treatment area. For example, the gantry 206 can be configured to rotate the therapy output 204 around an axis (“A”). Both the couch 216 and the radiation therapy output 204 can be independently moveable to other positions around the patient, such as moveable in transverse direction (“T”), moveable in a lateral direction (“L”), or as rotation about one or more other axes, such as rotation about a transverse axis (indicated as “R”). A controller communicatively connected to one or more actuators (not shown) may control the couch 216 movements or rotations in order to properly position the patient in or out of the radiation beam 208 according to a radiation therapy treatment plan. Both the couch 216 and the gantry 206 are independently moveable from one another in multiple degrees of freedom, which allows the patient to be positioned such that the radiation beam 208 can target the tumor precisely. The MLC may be integrated and included within gantry 206 to deliver the radiation beam 208 of a certain shape.

The coordinate system (including axes A, T, and L) shown in FIG. 2A can have an origin located at an isocenter 210. The isocenter can be defined as a location where the central axis of the radiation beam 208 intersects the origin of a coordinate axis, such as to deliver a prescribed radiation dose to a location on or within a patient. Alternatively, the isocenter 210 can be defined as a location where the central axis of the radiation beam 208 intersects the patient for various rotational positions of the radiation therapy output 204 as positioned by the gantry 206 around the axis A. As discussed herein, the gantry angle corresponds to the position of gantry 206 relative to axis A, although any other axis or combination of axes can be referenced and used to determine the gantry angle.

Gantry 206 may also have an attached imaging detector 214. The imaging detector 214 is preferably located opposite to the radiation source, and in an example, the imaging detector 214 can be located within a field of the radiation beam 208. The imaging detector 214 can be mounted on the gantry 206 (preferably opposite the radiation therapy output 204), such as to maintain alignment with the radiation beam 208. The imaging detector 214 rotates about the rotational axis as the gantry 206 rotates. In an example, the imaging detector 214 can be a flat panel detector (e.g., a direct detector or a scintillator detector). In this manner, the imaging detector 214 can be used to monitor the radiation beam 208 or the imaging detector 214 can be used for imaging the patient's anatomy, such as portal imaging. The control circuitry of the radiation therapy device 202 may be integrated within the radiotherapy system 100 or remote from it.

In an illustrative example, one or more of the couch 216, the therapy output 204, or the gantry 206 can be automatically positioned, and the therapy output 204 can establish the radiation beam 208 according to a specified dose for a particular therapy delivery instance. A sequence of therapy deliveries can be specified according to a radiation therapy treatment plan, such as using one or more different orientations or locations of the gantry 206, couch 216, or therapy output 204. The therapy deliveries can occur sequentially, but can intersect in a desired therapy locus on or within the patient, such as at the isocenter 210. A prescribed cumulative dose of radiation therapy can thereby be delivered to the therapy locus while damage to tissue near the therapy locus can be reduced or avoided.

FIG. 2B illustrates a radiation therapy device 202 that may include a combined Linac and an imaging system, such as a CT imaging system. The radiation therapy device 202 can include an MLC (not shown). The CT imaging system can include an imaging X-ray source 218, such as providing X-ray energy in a kiloelectron-Volt (keV) energy range. The imaging X-ray source 218 can provide a fan-shaped and/or a conical radiation beam 208 directed to an imaging detector 222, such as a flat panel detector. The radiation therapy device 202 can be similar to the system described in relation to FIG. 2A, such as including a radiation therapy output 204, a gantry 206, a couch 216, and another imaging detector 214 (such as a flat panel detector). The X-ray source 218 can provide a comparatively-lower-energy X-ray diagnostic beam, for imaging.

In the illustrative example of FIG. 2B, the radiation therapy output 204 and the X-ray source 218 can be mounted on the same rotating gantry 206, rotationally separated from each other by 90 degrees. In another example, two or more X-ray sources can be mounted along the circumference of the gantry 206, such as each having its own detector arrangement to provide multiple angles of diagnostic imaging concurrently. Similarly, multiple radiation therapy outputs 204 can be provided.

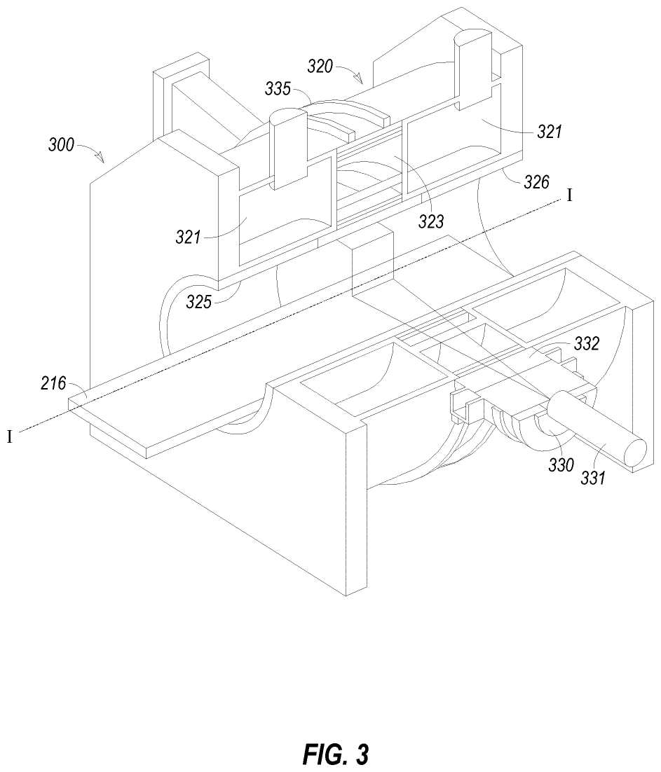

FIG. 3 depicts a radiation therapy system 300 that can include combining a radiation therapy device 202 and an imaging system, such as a magnetic resonance (MR) imaging system (e.g., known in the art as an MR-Linac) consistent with the disclosed examples. As shown, system 300 may include a couch 216, an image acquisition device 320, and a radiation delivery device 330. System 300 delivers radiation therapy to a patient in accordance with a radiotherapy treatment plan. In some examples, image acquisition device 320 may correspond to image acquisition device 170 in FIG. 1 that may acquire origin images of a first modality (e.g., an MRI image) or destination images of a second modality (e.g., an CT image).

Couch 216 may support a patient (not shown) during a treatment session. In some implementations, couch 216 may move along a horizontal translation axis (labelled “I”), such that couch 216 can move the patient resting on couch 216 into and/or out of system 300. Couch 216 may also rotate around a central vertical axis of rotation, transverse to the translation axis. To allow such movement or rotation, couch 216 may have motors (not shown) enabling the couch 216 to move in various directions and to rotate along various axes. A controller (not shown) may control these movements or rotations in order to properly position the patient according to a treatment plan.

In some examples, image acquisition device 320 may include an MRI machine used to acquire 2D or 3D MRI images of the patient before, during, and/or after a treatment session. Image acquisition device 320 may include a magnet 321 for generating a primary magnetic field for magnetic resonance imaging. The magnetic field lines generated by operation of magnet 321 may run substantially parallel to the central translation axis I. Magnet 321 may include one or more coils with an axis that runs parallel to the translation axis I. In some examples, the one or more coils in magnet 321 may be spaced such that a central window 323 of magnet 321 is free of coils. In other examples, the coils in magnet 321 may be thin enough or of a reduced density such that they are substantially transparent to radiation of the wavelength generated by radiotherapy device 330. Image acquisition device 320 may also include one or more shielding coils, which may generate a magnetic field outside magnet 321 of approximately equal magnitude and opposite polarity in order to cancel or reduce any magnetic field outside of magnet 321. As described below, radiation source 331 of radiation delivery device 330 may be positioned in the region where the magnetic field is cancelled, at least to a first order, or reduced.

Image acquisition device 320 may also include two gradient coils 325 and 326, which may generate a gradient magnetic field that is superposed on the primary magnetic field. Coils 325 and 326 may generate a gradient in the resultant magnetic field that allows spatial encoding of the protons so that their position can be determined. Gradient coils 325 and 326 may be positioned around a common central axis with the magnet 321 and may be displaced along that central axis. The displacement may create a gap, or window, between coils 325 and 326. In examples where magnet 321 can also include a central window 323 between coils, the two windows may be aligned with each other.

In some examples, image acquisition device 320 may be an imaging device other than an MRI, such as an X-ray, a CT, a CBCT, a spiral CT, a PET, a SPECT, an optical tomography, a fluorescence imaging, ultrasound imaging, radiotherapy portal imaging device, or the like. As would be recognized by one of ordinary skill in the art, the above description of image acquisition device 320 concerns certain examples and is not intended to be limiting.

Radiation delivery device 330 may include the radiation source 331, such as an X-ray source or a Linac, and an MLC 332. Radiation delivery device 330 may be mounted on a chassis 335. One or more chassis motors (not shown) may rotate the chassis 335 around the couch 216 when the couch 216 is inserted into the treatment area. In an example, the chassis 335 may be continuously rotatable around the couch 216, when the couch 216 is inserted into the treatment area. Chassis 335 may also have an attached radiation detector (not shown), preferably located opposite to radiation source 331 and with the rotational axis of the chassis 335 positioned between the radiation source 331 and the detector. Further, the device 330 may include control circuitry (not shown) used to control, for example, one or more of the couch 216, image acquisition device 320, and radiotherapy device 330. The control circuitry of the radiation delivery device 330 may be integrated within the system 300 or remote from it.

During a radiotherapy treatment session, a patient may be positioned on couch 216. System 300 may then move couch 216 into the treatment area defined by the magnet 321, coils 325, 326, and chassis 335. Control circuitry may then control radiation source 331, MLC 332, and the chassis motor(s) to deliver radiation to the patient through the window between coils 325 and 326 according to a radiotherapy treatment plan.

FIG. 2A, FIG. 2B, and FIG. 3 generally illustrate examples of a radiation therapy device configured to provide radiotherapy treatment to a patient, using a configuration where a radiation therapy output can be rotated around a central axis (e.g., an axis “A”). Other radiation therapy output configurations can be used. For example, a radiation therapy output can be mounted to a robotic arm or manipulator having multiple degrees of freedom. In yet another example, the therapy output can be fixed, such as located in a region laterally separated from the patient, and a platform supporting the patient can be used to align a radiation therapy isocenter with a specified target locus within the patient.

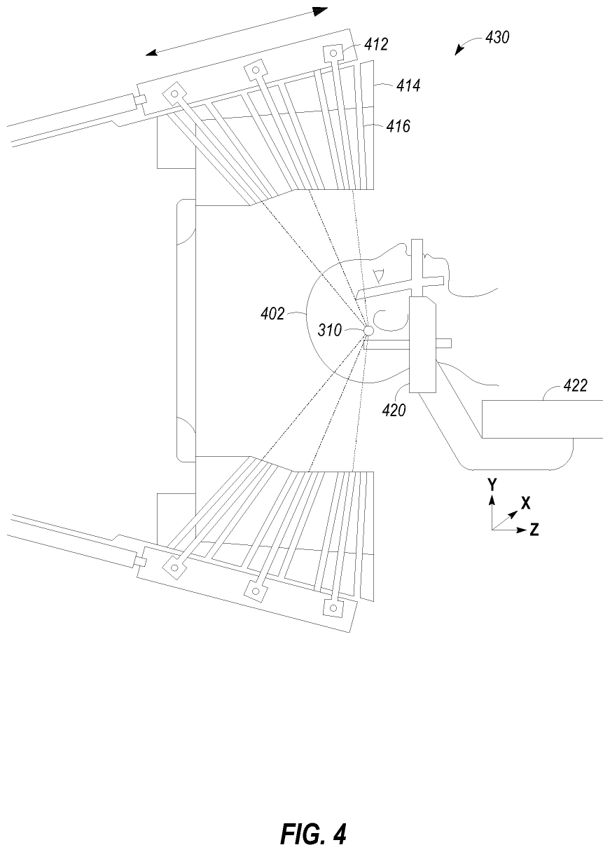

FIG. 4 illustrates a contrasting example of a Leksell Gamma Knife radiotherapy device 430, which provides such radiotherapy treatment by means of gamma radiation. As a brief overview of a Gamma Knife device, radiation is emitted from a large number of fixed radioactive sources and is focused by means of collimators, i.e. passages or channels for obtaining a beam of limited cross section, towards a defined target or treatment volume. Each of the sources provides a dose of gamma radiation which is insufficient to damage intervening tissue. However, tissue destruction occurs where the radiation beams from all or some radiation sources intersect or converge, causing the radiation to reach tissue-destructive levels. The point of convergence is hereinafter referred to as the “isocenter” but may also be referred to as a “focus point”.

As shown in FIG. 4, in a radiotherapy treatment session, a patient 402 may wear a coordinate frame 420 to keep stable the patient's body part (e.g., the head) undergoing surgery or radiotherapy. Coordinate frame 420 and a patient positioning system 422 may establish a spatial coordinate system, which may be used while imaging a patient or during radiation surgery. Radiotherapy device 430 may include a protective housing 414 to enclose a plurality of radiation sources 412. Radiation sources 412 may generate a plurality of radiation beams (e.g., beamlets) through beam channels 416. The plurality of radiation beams may be configured to focus on an isocenter 310 from different directions. While each individual radiation beam may have a relatively low intensity, isocenter 310 may receive a relatively high level of radiation when multiple doses from different radiation beams accumulate at isocenter 310. In certain examples, isocenter 310 may correspond to a target under surgery or treatment, such as a tumor.

Other types of radiotherapy devices (not illustrated) use protons and/or ions to deliver the radiotherapy treatment. The direction and shape of the radiation beam should be accurately controlled to ensure that the tumor receives the prescribed radiation dose, and the radiation from the beam should minimize damage to the surrounding healthy tissue, especially the organ(s) at risk (OARs). Thus, treatment planning is used to design and control radiation beam parameters, and a radiotherapy device effectuates a treatment by delivering a spatially varying dose distribution to the patient.

Treatment plan optimization for radiation therapy, such as for Gamma knife radiosurgery, aims at maximizing the dose delivered to the target volume within the patient (e.g. in treatment of tumors) at the same time as the dose delivered to adjacent normal tissues is minimized. In treatment plan optimization, the delivered radiation dose is mainly limited by two competing factors: the first one is delivering a high dose to the target volume and the second one is delivering low dose to the surrounding normal tissues. The treatment plan optimization is a process including optimizing the number of shots being used, the sector-collimator combinations, the shot times, and the position of the shot relative to the patient's anatomy (i.e. isocenter). Clearly, the irregularity and size of a target volume greatly influence the number of shots needed and the size of the shots being used to optimize the treatment.

Thus, for gamma knife radiotherapy, the selected isocenter locations and their corresponding shots for a given case constitutes a treatment plan.

The following provides a formal definition of a Pareto surface as used herein: Let Ω be an open subset of Rⁿ, or an n-dimensional surface, and let ƒ₁, ƒ₂, . . . , ƒₘ: Ω→R∪{∞} be extended-real valued functions. A point x̅∈Ω is called Pareto optimal, if there is no x∈Ω such that ƒᵢ(x)≤ƒᵢ(x̅) for all i=1 . . . m and ƒⱼ(x)<ƒⱼ(x) for some j. The collection of ƒᵢ(x̅) for all x̅, in the space spanned by ƒᵢ, is called a Pareto surface.

As provided herein, the optimization is of the form:

$${x}^{*} ∈ arg {min}_{x ∈ Ω} {\sum }_{i = 1}^{m} {w}_{i} {f}_{i} ( x ) \qquad ( Equation 3 )$$

Where the set of solutions x* is thus dependent on the weight vector w, i.e. x*=x*(w), and the i:th objective function value at the solution is ƒᵢ(x(w))≡ƒᵢ(w). For this kind of optimization problem, it can be shown that for any weight w the solution x is a non-dominated point x. Furthermore, the dimension of the Pareto surface is n−1, since the solution to Equation 3 is independent of scaling the weights by a scalar value (i.e., (w₁, w₂, . . . , wₙ) and λ(w₁, w₂, . . . , wₙ) gives the same solution).

The kind of generalized surfaces which are the subject of this optimization are referred to as “differentiable manifolds.” The surfaces locally look like a vector space in Rⁿ and one may define a mapping φ from an open set, chart, on the manifold to an open set in Rⁿ. The entire manifold is covered with a collection of charts which is called an atlas. The implication of the manifold locally looking like Rⁿ suggests the possibility of doing differentiable calculus on the manifold. For instance, if (U,φ) is an open set U on M with a mapping φ to Rⁿ and there is a second (V,ψ) open set V on M with a mapping ψ to Rⁿ and there is partly overlap between U and V (i.e. U∩V≠Ø), then ψφ⁻¹ must be differentiable. A simple example is a sphere that can be covered with minimum two charts (e.g., one excluding the north pole and the other excluding the south pole). Note that even though the sphere is embedded in three dimensions this is not necessary to know to geometrically describe its 2D surface. In fact, there are examples of 2D surfaces that cannot be embedded in 2D, e.g., the Klein bottle, but still can be described in terms of a finite number of 2D charts.

Depending on the nature of the optimization problem, a Pareto surface can be quite complicated, with several branches that can cross each other and be disconnected. However, assuming differentiability to 2:nd order of objective functions, Pareto surfaces are portions of (m−1) dimensional manifolds. In the case of convex objective functions the Pareto surface is simply a convex (m−1) dimensional manifold which can be covered by a few (possibly one) charts.

In an example, a regular submanifold to a manifold of dimension k is a subset S of the manifold M that locally looks like R^{k}. The submanifold inherits the differentiable structure of the manifold. The circle is a submanifold (one of an infinite number) of a sphere which in turn is a submanifold (one of an infinite number) of the 3D sphere x²+y²+z²+u²=R², and so on. In the case of a Pareto surface, a simple way to generate a submanifold with one dimension less is to fixate one of the weights and then optimize for the rest. This can of course be extended to generate submanifolds of dimension n−2, n−3, . . . 1. By just varying one of the weights, the submanifold is a line along which the objective functions vary. Choosing a fixed a weight, call it w i=1 . . . n−1, we obtain a single point, p^{s}, on the Pareto surface, P. By varying each element in the weight at a time, wᵢ=wᵢ^{s}+τ, we get n−1 lines on the Pareto surface all passing through p^{s}. These lines form a grid or a coordinate system on the Pareto surface close to p^{s}. Very close to p^{s} these lines become tangent vectors to the surface. The collection of all tangent vectors at p^{s}, called the tangent space, is denoted Tₚ_{^{s}} P. The union of all tangent spaces of the manifold is called the tangent bundle TP=∪ₚ_{^{s}} Tₚ_{^{s}} P. Note that in general Tₚ_{^{s}} P≠T_{q}_{^{s}} P if p^{s}≠q^{s}.

Accordingly, references to navigation or “surfing” used herein, refer to the concept of starting on a specific point p^{s}, on P, and then explore how the cost function values change as we go along a given direction. Because of the high dimensionality of the Pareto surface, it is computationally more tractable and easier to visualize how the submanifold changes than the entire Pareto surface. Moreover, the direction in which to move could be unknown and becomes a part of the navigation. For instance, it could be of interest to minimize one objective while controlling the degradation of the others. Therefore, at each point p^{s} the tangent vector in Tₚ_{^{s}} P is found that minimizes the said objective. By moving along the tangent vector infinitesimally a nearby point is reached. The process is repeated until the user deems the other objectives to be too poor. As detailed below, this simple navigation can be formulated as an ordinary differential equation and the solution is a path on the Pareto surface.

FIG. 5 provides a high-level view of radiotherapy treatment planning workflow operations. Specifically, this workflow includes the use of pareto navigation and exploration as part of a radiotherapy problem optimization process, which evaluates and formulates Pareto-optimal solutions for a radiotherapy problem.

The operations in FIG. 5, in more detail, illustrate how radiotherapy problem information 510, for a treatment of a human subject, is provided with the definition of information such as target areas of treatment 512 and organ at risk areas 514 (or, defined areas of healthy tissue). Other information relevant to the radiotherapy problem may include radiotherapy machine information 520 such as machine capabilities 522. The parameters (criteria) of the radiotherapy problem therefore may correspond to clinical preferences, anatomical areas, detailed definitions of areas to receive (or not to receive) treatment, and the like.

Given these parameters and information, a process of radiotherapy problem solving 530 is performed to generate multiple radiotherapy problem solutions 540. These solutions may be generated using a variety of problem solvers, to produce an initial set of Pareto-optimal radiotherapy solutions. As noted above, this set of Pareto-optimal radiotherapy solutions may constitute or define a Pareto surface, which is explored with the following navigation techniques.

The navigation of the pareto-optimal solutions 550 may include the following functions or capabilities:
Directional exploration 552, which enables conversion, portrayal, and navigation of solutions within a representation of a solution space;
Selection functionality 554, which enables selection and identification of particular parameter weights (i.e., parameter values) within a solution space;
Restriction functionality 556, which enables the exclusion of particular parameter weights (i.e., parameter values) within a solution space; and
Identification functionality 558, which enables an identification of a new, selected solution (i.e., an additional solution) at a particular location in the solution space.

The new, selected solution provides a specific combination of weight values which can be used for the generation of radiotherapy treatment data, and ultimately, the generation of a particular radiotherapy treatment plan with operation 560. The generation of the radiotherapy treatment plan with operation 560 (and, related selection or modification of the radiotherapy solution) may be dependent on machine capabilities 522. Finally, radiotherapy treatment may be delivered using the generated treatment plan (and treatment plan-related data) with operation 570.

In an example, the navigation and directional exploration of the pareto-optimal solutions is performed with the use of a submanifold. In an example, the submanifold is one-dimensional, which implies that coordinate charts are one-dimensional paths in weight space, and the generated solutions to the MCO problem belong to the zero level-set of the optimality conditions. The navigation then reduces to solving an ordinary differential equation (ODE) based on a directional derivative. There are well-developed solution methods for ODEs that can, for instance, track the accuracy of the solution and determine adaptively when the function (and possibly derivatives) needs to be evaluated to achieve a certain level of accuracy. In this setting, such queries may amount to solving the optimization problem, which can preferentially be done using a warm start method considering that the ODE solver is likely able to produce an accurate initial guess.

It will be understood that three different but related spaces are involved in the exploration of the solutions:
i) The vector of objective function values, ƒ(x)=(ƒ₁ (x), . . . , ƒₘ(x)), exists in objective space, which is typically a subset of R^{m} (with m being the number of objectives).
ii) The weights w exist in weight space. Technically, the multicriteria optimization problem is a vector optimization problem that seeks to minimize the vector of objective functions with respect to some cone K, and the weight space is the set of nonnegative vectors in the dual cone K*.
iii) The decision variables x, interchangeably referred to herein as optimization variables, exist in decision variable space, which is an n-dimensional space (typically a subset of Rⁿ). Here, n>m, but because of the direct correspondence between the m-dimensional weights and solutions, the set of Pareto-optimal solutions (in decision variable space) is an m−1-dimensional manifold embedded in the much higher-dimensional decision variable space.

A “solution” as used herein therefore may refer to a vector of decision variables x*, or the corresponding vector of objective function values ƒ(x*), or both. Similarly, the concept of Pareto surface may refer to the set of Pareto-optimal solutions in an objective space, decision variable space, or both.

FIG. 6 provides an example workflow for identification and exploration of additional pareto-optimal solutions. In an example, this workflow may be implemented by the navigation of pareto-optimal solutions 550 (and sub-functionality 552, 554, 556, 558) as part of a radiotherapy treatment planning and optimization process. However, this workflow may also be implemented as a standalone evaluative process, or other aspects of solution planning and exploration.

The workflow begins at operation 610, by obtaining a pareto surface (or, a sufficiently large set) of potential solutions to a radiotherapy problem. This solution space provides a plurality of solutions to a radiotherapy problem (a multicriteria optimization problem) that is adjustable via weights of a plurality of criteria. The solutions of the radiotherapy problem provide a subset of the Pareto surface (where each Pareto-optimal point could in principle be found, one at the time, by solving the radiotherapy problem for each choice of weights).

At operation 620, a path-connected set of weights is defined, within a submanifold of the Pareto surface. The submanifold space has fewer dimensions than a cardinality of the plurality of weights used by the criteria of the radiotherapy problem. Cardinality, as used herein, can be understood with the following example: If there are N solutions (e.g., hundreds of solutions), and each solution corresponds to M weights (e.g., a dozen or less weights), then the dimension of the submanifold is less than M (and can be at most M−1, since the solutions are invariant to the scaling of the objective function).

In an example, the submanifold space provides a one-dimensional path for a navigation on a path. However, in another example, the submanifold space may also be navigated with use of a multi-dimensional surface. Consider a Pareto surface with N solutions (e.g., hundreds of solutions), with each solution corresponding to M weights (e.g., a dozen or less weights). In this setting, the dimension of the submanifold is less than M (and can be at most M−1 since the solutions are invariant to the scaling of the objective function).

At operation 630, the navigation in the submanifold, is reduced to an ordinary differential equation (ODE) based on a parameterized direction of the path. In an example, the differential equation is formulated on the path-connected set of weights based on first-order optimality conditions. For instance, navigation of the one-dimensional path may be started from a Pareto-optimal initial point, as the navigation reduces to an ordinary differential equation based on a directional derivative. For a constrained optimization problem, compliance with Karush-Kuhn-Tucker (KKT) conditions are a first-order optimality condition.

In an example, the exploration is provided by solving a boundary value problem, where an initial point and a final point are provided for the navigation of the one-dimensional path. For solution of a boundary value problem, the navigation interpolates between the initial point and the final point. In another example, the exploration is provided by solving an initial value problem, where an initial point is provided for the navigation of the one-dimensional path. In this example, the navigation operates until meeting a predetermined stopping condition. The predetermined stopping condition may be: time, distance, or maximum acceptable deterioration of a clinical metric. For instance, the maximum acceptable deterioration of a clinical metric, may also involve use of a simultaneous localization and mapping (SLAM) method to improve a first clinical metric while minimizing deterioration of a second clinical metric.

In some examples, the radiotherapy problem is a constrained problem, and at least a portion of the manifold space has a non-differentiable portion. (This occurs when the Pareto surface may be non-differentiable where the active set, i.e. the set of constraints that hold with equality, changes), because the Optional operations 640 and 650 address these scenarios. At operation 640, non-differentiable areas are identified. At operation 650, constraints are removed from these non-differentiable areas. In one example, a least-squares method is used to smoothly approximate the non-differentiable portion of the manifold space. In another example, a barrier formulation is used to convert the constrained problem into an unconstrained problem, by including constraints as terms in an objective function that associates violations of the constraints with penalties.

At operation 660, differentiation is performed, by solving the differential equation (the ODE) to generate additional pareto-optimal solutions. In an example, such differentiation may be performed by automated differentiation techniques. In another example, such differentiation may be performed analytically, such as evaluating the derivatives from vector-Jacobian products (thus, circumventing the need to compute and store the full Jacobian matrix).

At operation 670, exploration of the newly generated (additional) pareto-optimal solutions occurs. Here, such exploration may occur using coordinate charts that is provided beforehand or dynamically, which is analogous to the exploration being determined by open or closed-loop control, respectively. As noted above, such explorations be controlled based on solution to a boundary value problem or an initial value problem, and accompanying stopping conditions.

At evaluation 680, a determination is made whether a satisfactory solution is found from the additional pareto-optimal solutions. This determination may be made based on evaluative criteria or user input. If a satisfactory solution is found, the solution is utilized in the radiotherapy plan at operation 690. If a satisfactory solution is not found, then operations to perform additional exploration and path finding are repeated (e.g., repeating operations 620-670).

As discussed above, a non-differentiable portion of the Pareto surface may be encountered, which is resolved through smooth approximations and barrier constraints. Constraints will restrict the feasible set of solutions and therefore change the Pareto surface compared to a non-constraint problem. For some of the weight combinations the “un-constrained solutions” will still be viable in the constrained case, but for some other combinations the Pareto surface will be modified. On the Pareto surface there is not a smooth connection of the tangent space from the un-changed parts of the Pareto surface to parts of the Pareto surface modified by the constraints, which means that navigation methods based on solving ordinary or partial differential equations will fail. Close to the “ridge”, one can modify the Pareto surface to make it vary smoothly, thus giving a smooth connection of the tangent spaces implying that the above-mentioned navigation methods will apply.

Another way of formulating an optimization problem with constraints is to introduce a barrier function into the objective function. The idea is that the closer the solution is to violate the constraint, the higher the value of the barrier function. If the constraint is of the form Ax≤b a typical barrier function is:

$$Φ ( x ) = \{ \begin{matrix}- log ( b - Ax ) if Ax < b \\ + ∞ otherwise\end{matrix}$$

The high cost when being close to the constraint (and infinite at the constraint and beyond) leads to solutions which do not violate the constraint. Replacing the constraint with the barrier penalization term leads to a smooth differentiable Pareto surface, since the optimization problem consists of smooth and differentiable terms. The barrier function accomplishes the same thing as the smooth approximations but without the need to explicitly introduce terms to smooth the Pareto surface.

Although not directly depicted in the drawings discussed herein, various forms of user interfaces or representations may be provided to enable a representation of a solution space to the radiotherapy problem, based on the plurality of pareto-optimal solutions. This may be provided in a graphical user interface having functionality to configure the treatment plan, and to receive and output data related to the treatment plan. For instance, information associated with a solution to the radiotherapy problem, for a particular set of parameters, may be displayed. User interaction may be obtained in such a user interface for modifying the particular set of parameters. As will be understood, a variety of interactive Pareto navigation functions may be provided in the user interface, such as an approximation of the Pareto surface that lets a user explore estimates of solutions corresponding to new or different parameter values that have not been previously evaluated.

It will be understood that the Pareto optimal plans generated with such techniques can be considered as points on a Pareto surface. The more points that are generated, the better the description of the surface. When a user explores the Pareto surface, the user performs some type of interpolation between existing calculated points in order to select and use a solution. Plausibly, the user will ultimately develop and choose a plan that is not necessarily one of the calculated points.

In some examples, all the Pareto-optimal solutions, or at least a substantial subset of the pareto-optimal solutions, may be generated and evaluated before a particular plan or solution is selected by a user. Thus, the ultimately chosen plan may be a plan “in between” two generated points on the pareto-surface. Using the techniques discussed herein, navigating the Pareto surface can be performed quickly and efficiently.

FIG. 7 illustrates a representation 700 of exploration in a Pareto surface. Here, three dimensions are shown: a first axis 710 representing the total irradiation time values of a treatment (e.g., a measurement of beam-on-time (BOT) for a particular radiotherapy solution); a second axis 720 representing values of selectivity (i.e. the fraction of volume of dose being equal or higher than the planned treatment dose limited to the target volume, defining selectivity between areas inside and outside of the region of interest for a particular radiotherapy solution); and a third axis 730 representing coverage of the radiotherapy solution (i.e. the fraction of target volume receiving equal or more than the planned dose, defining a coverage of the area to treat for a particular radiotherapy solution). The modeling and rendering of this representation 700 with the three dimensions allows exploration of the Pareto surface along a one-dimensional path (path 740).

Here, the exploration along the path 740 demonstrates that by accepting a small degradation (e.g., a maximum of 2%) in quality (e.g., coverage and/or selectivity), a treatment plan can be identified that has 25% shorter treatment time (e.g., beam-on-time). The path is along the steepest negative gradient in the treatment time dimension fulfilling the requirements of maximum allowed degradation in quality.

FIGS. 8A-8D provide further representations of exploration in a Pareto surface. First, FIG. 8A illustrates a collection of Pareto optimal points for an optimization case with four objectives rendering the Pareto surface in 3D. The shading (darkness in shading) and the size of the filled circles are proportional to the value of the fourth objective term.

FIG. 8B illustrates a result of smoothing a part of the Pareto surface from FIG. 8A. As discussed above, smoothing provides a mechanism for efficient Pareto surfing.

FIG. 8C illustrates a result of fixing the fourth objective to a certain value, to produce a 2D-submanifold. In this drawing, interpolation has been applied to illustrate the surface.

FIG. 8D illustrates a result of fixating multiple objectives (f3 and f4) to specific values. Here, this results in a representation of a 1D submanifold, representing a 1D path on the 3D Pareto surface.

FIG. 9 illustrates a flowchart 900 of a method of radiotherapy treatment planning based on the techniques discussed above. For instance, the following features of flowchart 900 may be integrated or adapted with the navigation and optimization operations discussed with reference to FIG. 5, the solution exploration operations discussed with reference to FIG. 6, and the path exploration operations discussed with reference to FIG. 7 and FIGS. 8A-8D.

Operation 910 begins with operations to obtain a radiotherapy problem, with the radiotherapy problem defining various parameters for delivery of a radiotherapy treatment from a radiotherapy machine. Such a radiotherapy problem may be adjustable via parameters, which are optionally received with a request to solve the radiotherapy problem.

Operation 920 proceeds with operations to produce solutions for a radiotherapy problem, which may be performed for individual problems until an entire Pareto surface or frontier (or, a sufficient portion of the Pareto surface or frontier) of solutions are identified. Although not shown, the treatment plan optimization operations may be based on dose, geometry, imaging, machine learning, radiobiology, or other relevant factors. A variety of solution optimizers and solutions methods may be used to produce the available solutions.

Operation 930 proceeds with a process to identify an additional radiotherapy solution from an exploration of the pareto-optimal radiotherapy solutions. This may be provided from sub-operations including: at operation 932, establishing a submanifold space from the solutions in the pareto surface; at operation 934, producing additional weights in the submanifold space, based on derivatives of optimality conditions; at operation 936, performing navigation along a path in the submanifold space to arrive at the additional solution; and at operation 938, selecting a set of weights from the additional solution.

At operation 940, the additional solution is used to generate treatment plan data, and as applicable, this additional solution is further optimized and implemented as part of a treatment plan. For instance, the additional solution may provide a warm start to identify a solution used for the treatment plan, with additional optimization being received and implemented to the additional solution to create the treatment plan. Consistent with the examples above, the treatment plan data for the radiotherapy treatment may include a set of treatment delivery parameters corresponding to capabilities and operational controls of the radiotherapy machine.

FIG. 10 illustrates a block diagram of an example of a machine 1000 on which one or more of the methods as discussed herein can be implemented. In one or more examples, one or more items of the radiotherapy processing computing system 110 can be implemented by the machine 1000. In alternative examples, the machine 1000 operates as a standalone device or may be connected (e.g., networked) to other machines. In one or more examples, the radiotherapy processing computing system 110 can include one or more of the items of the machine 1000. In a networked deployment, the machine 1000 may operate in the capacity of a server or a client machine in server-client network environment, or as a peer machine in a peer-to-peer (or distributed) network environment. The machine may be a personal computer (PC), server, a tablet, smartphone, a web appliance, edge computing device, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein.

The example machine 1000 includes processing circuitry or processor 1002 (e.g., a CPU, a graphics processing unit (GPU), an ASIC, circuitry, such as one or more transistors, resistors, capacitors, inductors, diodes, logic gates, multiplexers, buffers, modulators, demodulators, radios (e.g., transmit or receive radios or transceivers), sensors 1021 (e.g., a transducer that converts one form of energy (e.g., light, heat, electrical, mechanical, or other energy) to another form of energy), or the like, or a combination thereof), a main memory 1004 and a static memory 1006, which communicate with each other via a bus 1008. The machine 1000 (e.g., computer system) may further include a video display device 1010 (e.g., a liquid crystal display (LCD) or a cathode ray tube (CRT)). The machine 1000 also includes an alphanumeric input device 1012 (e.g., a keyboard), a user interface (UI) navigation device 1014 (e.g., a mouse), a disk drive or mass storage unit 1016, a signal generation device 1018 (e.g., a speaker), and a network interface device 1020.

The disk drive unit 1016 includes a machine-readable medium 1022 on which is stored one or more sets of instructions and data structures (e.g., software) 1024 embodying or utilized by any one or more of the methodologies or functions described herein. The instructions 1024 may also reside, completely or at least partially, within the main memory 1004 and/or within the processor 1002 during execution thereof by the machine 1000, the main memory 1004 and the processor 1002 also constituting machine-readable media.

The machine 1000 as illustrated includes an output controller 1029. The output controller 1029 manages data flow to/from the machine 1000. The output controller 1028 is sometimes called a device controller, with software that directly interacts with the output controller 1028 being called a device driver.

While the machine-readable medium 1022 is shown in an example to be a single medium, the term “machine-readable medium” may include a single medium or multiple media (e.g., a centralized or distributed database, and/or associated caches and servers) that store the one or more instructions or data structures. The term “machine-readable medium” shall also be taken to include any tangible medium that is capable of storing, encoding or carrying instructions for execution by the machine and that cause the machine to perform any one or more of the methodologies of the present disclosure, or that is capable of storing, encoding or carrying data structures utilized by or associated with such instructions. The term “machine-readable medium” shall accordingly be taken to include, but not be limited to, solid-state memories, and optical and magnetic media. Specific examples of machine-readable media include non-volatile memory, including by way of example semiconductor memory devices, e.g., Erasable Programmable Read-Only Memory (EPROM), EEPROM, and flash memory devices; magnetic disks such as internal hard disks and removable disks; magneto-optical disks; and CD-ROM and DVD-ROM disks.

The instructions 1024 may further be transmitted or received over a communications network 1026 using a transmission medium. The instructions 1024 may be transmitted using the network interface device 1020 and any one of a number of well-known transfer protocols (e.g., HTTP). Examples of communication networks include a LAN, a WAN, the Internet, mobile telephone networks, Plain Old Telephone (POTS) networks, and wireless data networks (e.g., Wi-Fi and 4G/5G data networks). The term “transmission medium” shall be taken to include any intangible medium that is capable of storing, encoding or carrying instructions for execution by the machine, and includes digital or analog communications signals or other intangible media to facilitate communication of such software.

As used herein, “communicatively coupled between” means that the entities on either of the coupling must communicate through an item therebetween and that those entities cannot communicate with each other without communicating through the item.

### Additional Notes

The above detailed description includes references to the accompanying drawings, which form a part of the detailed description. The drawings show, by way of illustration but not by way of limitation, specific embodiments in which the disclosure can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a,” “an,” “the,” and “said” are used when introducing elements of aspects of the disclosure or in the embodiments thereof, as is common in patent documents, to include one or more than one or more of the elements, independent of any other instances or usages of “at least one” or “one or more.” In this document, the term “or” is used to refer to a nonexclusive or, such that “A or B” includes “A but not B,” “B but not A,” and “A and B,” unless otherwise indicated.

In the appended claims, the terms “including” and “in which” are used as the plain-English equivalents of the respective terms “comprising” and “wherein.” Also, in the following claims, the terms “comprising,” “including,” and “having” are intended to be open-ended to mean that there may be additional elements other than the listed elements, such that after such a term (e.g., comprising, including, having) in a claim are still deemed to fall within the scope of that claim. Moreover, in the following claims, the terms “first,” “second,” and “third,” and so forth, are used merely as labels, and are not intended to impose numerical requirements on their objects.

Embodiments of the disclosure may be implemented with computer-executable instructions. The computer-executable instructions (e.g., software code) may be organized into one or more computer-executable components or modules. Aspects of the disclosure may be implemented with any number and organization of such components or modules. For example, aspects of the disclosure are not limited to the specific computer-executable instructions or the specific components or modules illustrated in the figures and described herein. Other embodiments of the disclosure may include different computer-executable instructions or components having more or less functionality than illustrated and described herein.

Method examples (e.g., operations and functions) described herein can be machine or computer-implemented at least in part (e.g., implemented as software code or instructions). Some examples can include a computer-readable medium or machine-readable medium encoded with instructions operable to configure an electronic device to perform methods as described in the above examples. An implementation of such methods can include software code, such as microcode, assembly language code, a higher-level language code, or the like (e.g., “source code”). Such software code can include computer-readable instructions for performing various methods (e.g., “object” or “executable code”). The software code may form portions of computer program products. Software implementations of the embodiments described herein may be provided via an article of manufacture with the code or instructions stored thereon, or via a method of operating a communication interface to send data via a communication interface (e.g., wirelessly, over the internet, via satellite communications, and the like).

Further, the software code may be tangibly stored on one or more volatile or non-volatile computer-readable storage media during execution or at other times. These computer-readable storage media may include any mechanism that stores information in a form accessible by a machine (e.g., computing device, electronic system, and the like), such as, but are not limited to, floppy disks, hard disks, removable magnetic disks, any form of magnetic disk storage media, CD-ROMS, magnetic-optical disks, removable optical disks (e.g., compact disks and digital video disks), flash memory devices, magnetic cassettes, memory cards or sticks (e.g., secure digital cards), RAMs (e.g., CMOS RAM and the like), recordable/non-recordable media (e.g., read only memories (ROMs)), EPROMS, EEPROMS, or any type of media suitable for storing electronic instructions, and the like. Such computer-readable storage medium is coupled to a computer system bus to be accessible by the processor and other parts of the OIS.

In an embodiment, the computer-readable storage medium may have encoded a data structure for treatment planning, wherein the treatment plan may be adaptive. The data structure for the computer-readable storage medium may be at least one of a Digital Imaging and Communications in Medicine (DICOM) format, an extended DICOM format, an XML format, and the like. DICOM is an international communications standard that defines the format used to transfer medical image-related data between various types of medical equipment. DICOM RT refers to the communication standards that are specific to radiation therapy.

In various embodiments of the disclosure, the method of creating a component or module can be implemented in software, hardware, or a combination thereof. The methods provided by various embodiments of the present disclosure, for example, can be implemented in software by using standard programming languages such as, for example, C, C++, C#, Java, Python, CUDA programming, and the like; and combinations thereof. As used herein, the terms “software” and “firmware” are interchangeable, and include any computer program stored in memory for execution by a computer.

A communication interface includes any mechanism that interfaces to any of a hardwired, wireless, optical, and the like, medium to communicate to another device, such as a memory bus interface, a processor bus interface, an Internet connection, a disk controller, and the like. The communication interface can be configured by providing configuration parameters and/or sending signals to prepare the communication interface to provide a data signal describing the software content. The communication interface can be accessed via one or more commands or signals sent to the communication interface.

The present disclosure also relates to a system for performing the operations herein. This system may be specially constructed for the required purposes, or it may comprise a general purpose computer selectively activated or reconfigured by a computer program stored in the computer. The order of execution or performance of the operations in embodiments of the disclosure illustrated and described herein is not essential, unless otherwise specified. That is, the operations may be performed in any order, unless otherwise specified, and embodiments of the disclosure may include additional or fewer operations than those disclosed herein. For example, it is contemplated that executing or performing a particular operation before, contemporaneously with, or after another operation is within the scope of aspects of the disclosure.

In view of the above, it will be seen that the several objects of the disclosure are achieved and other advantageous results attained. Having described aspects of the disclosure in detail, it will be apparent that modifications and variations are possible without departing from the scope of aspects of the disclosure as defined in the appended claims. As various changes could be made in the above constructions, products, and methods without departing from the scope of aspects of the disclosure, it is intended that all matter contained in the above description and shown in the accompanying drawings shall be interpreted as illustrative and not in a limiting sense.

The above description is intended to be illustrative, and not restrictive. For example, the above-described examples (or one or more aspects thereof) may be used in combination with each other. In addition, many modifications may be made to adapt a particular situation or material to the teachings of the disclosure without departing from its scope. While the dimensions, types of materials and coatings described herein are intended to define the parameters of the disclosure, they are by no means limiting and are exemplary embodiments. Many other embodiments will be apparent to those of skill in the art upon reviewing the above description. The scope of the disclosure should, therefore, be determined with reference to the appended claims, along with the full scope of equivalents to which such claims are entitled.

Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the disclosure should be determined with reference to the appended claims, along with the full scope of equivalents to which such claims are entitled. Further, the limitations of the following claims are not written in means-plus-function format and are not intended to be interpreted based on 35 U.S.C. § 112, sixth paragraph, unless and until such claim limitations expressly use the phrase “means for” followed by a statement of function void of further structure.

The Abstract is provided to comply with 37 C.F.R. § 1.72(b), to allow the reader to quickly ascertain the nature of the technical disclosure. It is submitted with the understanding that it will not be used to interpret or limit the scope or meaning of the claims.

1. A computer-implemented method for radiotherapy treatment planning, the method comprising:
obtaining a plurality of solutions, defined in a manifold space, of a radiotherapy problem for providing radiotherapy treatment, wherein the radiotherapy problem is a multicriteria optimization problem, and wherein each of the plurality of solutions has a plurality of weights used to adjust a plurality of criteria for the multicriteria optimization problem;
exploring the plurality of solutions for the multicriteria optimization problem to identify an additional solution in a submanifold space, the exploring comprising:
establishing the submanifold space from the manifold space, the submanifold space representing the plurality of solutions in fewer dimensions than the plurality of weights;
producing additional sets of weights in the submanifold space based on derivatives of first-order optimality conditions of the radiotherapy problem, the derivatives determined with respect to the plurality of weights; and
performing navigation in the submanifold space to arrive at the additional solution, the additional solution corresponding to one of the additional sets of weights; and
generating treatment plan parameters based on the additional solution, wherein the treatment plan parameters are used in a treatment plan for delivery of the radiotherapy treatment via a radiotherapy machine.

2. The method of claim 1, wherein the navigation is performed in the submanifold space using a one-dimensional path.

3. The method of claim 2, wherein the navigation of the one-dimensional path is performed starting from a Pareto-optimal initial point, and wherein the navigation reduces to finding the one-dimensional path by solving an ordinary differential equation based on a directional derivative.

4. The method of claim 2, wherein to identify the additional solution solves a boundary value problem, wherein an initial point and a final point are provided for the navigation of the one-dimensional path, and wherein the navigation interpolates between the initial point and the final point.

5. The method of claim 2, wherein to identify the additional solution solves an initial value problem, wherein an initial point is provided for the navigation of the one-dimensional path, and wherein the navigation operates until meeting a predetermined stopping condition.

6. The method of claim 5, wherein the predetermined stopping condition is: time, distance, or maximum acceptable deterioration of a clinical metric.

7. The method of claim 6, wherein the predetermined stopping condition is maximum acceptable deterioration of a clinical metric, wherein a simultaneous localization and mapping (SLAM) method is used to perform the navigation on the one-dimensional path, wherein the SLAM method identifies improvement to a first clinical metric while minimizing deterioration of a second clinical metric.

8. The method of claim 1, wherein the additional sets of weights in the submanifold space correspond to a level-set of the optimality conditions of the radiotherapy problem.

9. The method of claim 1, wherein the derivatives are provided by automated differentiation.

10. The method of claim 1, wherein at least a portion of the manifold space has a non-differentiable portion.

11. The method of claim 10, wherein a least-squares method is used to smoothly approximate the non-differentiable portion of the manifold space.

12. The method of claim 10, wherein a barrier formulation is used to convert a constrained problem into an unconstrained problem, by including constraints as terms in an objective function that associates violations of the constraints with penalties.

13. The method of claim 1, wherein the plurality of criteria for the radiotherapy problem correspond to clinical preferences, and wherein at least one criterion in the plurality of criteria relates to a particular anatomical area to receive the radiotherapy treatment from the radiotherapy machine.

14. The method of claim 1, further comprising generating a representation of a solution space based on the plurality of solutions to the radiotherapy problem, wherein the solution space is a Pareto surface comprising a set of Pareto optimal solutions.

15. The method of claim 1, further comprising:
generating a display of a graphical user interface, the graphical user interface configured to provide functionality to configure the treatment plan; and
displaying, within the graphical user interface, information associated with the additional solution.

16. The method of claim 1, further comprising:
receiving a selection of the additional solution;
wherein the treatment plan parameters are generated based on the selection of the additional solution.

17. The method of claim 16, wherein the additional solution provides a warm start to identify a solution used for the treatment plan, with the method further comprising:
receiving an optimization to the additional solution;
wherein the treatment plan parameters are generated based on the optimization to the additional solution.

18. The method of claim 1, wherein the treatment plan parameters for the radiotherapy treatment comprises a set of treatment delivery parameters corresponding to capabilities of the radiotherapy machine.

19. The method of claim 18, wherein the treatment plan is used to provide the radiotherapy treatment with a Gamma knife, and wherein the set of treatment delivery parameters comprises a set of isocenters used for delivery of the radiotherapy treatment.

20. The method of claim 18, wherein the set of treatment delivery parameters further comprises timing for delivery of the radiotherapy treatment and a collimator sequence for the delivery of the radiotherapy treatment.

21. The method of claim 18, wherein the treatment plan is used to provide the radiotherapy treatment with a Linac or magnetic resonance (MR)-Linac radiotherapy machine.

22. The method of claim 21, wherein the treatment plan is used to provide the radiotherapy treatment with Volumetric-modulated arc therapy (VMAT) or Intensity modulated radiation therapy (IMRT), and wherein the set of treatment delivery parameters comprises: a set of arc control points for one or more arcs, fluence fields, gantry speed, and dose rate along the one or more arcs.

23. A non-transitory computer-readable storage medium comprising computer-readable instructions for radiotherapy treatment planning, wherein the instructions, when executed with a computing machine, cause the computing machine to:
obtain a plurality of solutions, defined in a manifold space, of a radiotherapy problem for providing radiotherapy treatment, wherein the radiotherapy problem is a multicriteria optimization problem, and wherein each of the plurality of solutions has a plurality of weights used to adjust a plurality of criteria for the multicriteria optimization problem;
explore the plurality of solutions for the multicriteria optimization problem to identify an additional solution in a submanifold space, the exploring comprising:
establishing the submanifold space from the manifold space, the submanifold space representing the plurality of solutions in fewer dimensions than the plurality of weights;
producing additional sets of weights in the submanifold space based on derivatives of first-order optimality conditions of the radiotherapy problem, the derivatives determined with respect to the plurality of weights; and
performing navigation in the submanifold space to arrive at the additional solution, the additional solution corresponding to one of the additional sets of weights; and
generate treatment plan parameters based on the additional solution, wherein the treatment plan parameters are used in a treatment plan for delivery of the radiotherapy treatment via a radiotherapy machine.

24. A computing system configured for radiotherapy treatment planning, the computing system comprising:
one or more memory devices to store data of a radiotherapy problem for providing radiotherapy treatment to a human subject from a radiotherapy treatment machine; and
one or more processors configured to perform operations to:
obtain a plurality of solutions to the radiotherapy problem, defined in a manifold space, wherein the radiotherapy problem is a multicriteria optimization problem, and wherein each of the plurality of solutions has a plurality of weights used to adjust a plurality of criteria for the multicriteria optimization problem;
explore the plurality of solutions for the multicriteria optimization problem to identify an additional solution in a submanifold space, with operations to:
establish the submanifold space from the manifold space, the submanifold space representing the plurality of solutions in fewer dimensions than the plurality of weights;
produce additional sets of weights in the submanifold space based on derivatives of first-order optimality conditions of the radiotherapy problem, the derivatives determined with respect to the plurality of weights; and
perform navigation in the submanifold space to arrive at the additional solution, the additional solution corresponding to one of the additional sets of weights; and
generate treatment plan parameters based on the additional solution, wherein the treatment plan parameters are used in a treatment plan for delivery of the radiotherapy treatment via a radiotherapy machine.

25.-45. (canceled)
